(19) United States
(12) Patent Application Publication
(10) Pub. No.: US 2026/0015332 A1
(43) Pub. Date: Jan. 15, 2026

(54) SYNTHESIS METHOD AND APPLICATION OF BENZOTHIASELENAZOLE-1-OXIDE COMPOUND AND DERIVATIVE THEREOF

(71) Applicant: GUANGZHOU MEDICAL UNIVERSITY, Guangdong (CN)

(72) Inventors: Wei YI, Guangdong (CN); Zhi ZHOU, Guangdong (CN); Liexin WU, Guangdong (CN)

(73) Assignee: GUANGZHOU MEDICAL UNIVERSITY, Guangdong (CN)

(21) Appl. No.: 18/993,576

(22) PCT Filed: Jan. 10, 2023
(86) PCT No.: PCT/CN2023/071718

§ 371 Date: Jan. 13, 2025

(30) Foreign Application Priority Data
Oct. 24, 2022 (CN) 202211304995.1

(51) Int. Cl. C07D 293/10

(52) U.S. Cl. CPC C07D 293/10

(57) ABSTRACT

The present invention discloses the synthetic methods and the corresponding applications of a benzothiaselenazole-1-oxide compound and derivatives thereof, in which with sulfoximine and elemental selenium as starting materials, a series of benzothiaselenazole-1-oxide compounds has been synthesized through rhodium-catalyzed direct C—H functionalization reaction. Furthermore, with sulfoximine and elemental selenium as starting materials, a chiral benzothiaselenazole-1-oxide compound is synthesized through direct C—H functionalization reaction by virtue of a chiral phosphoric acid ligand. The present invention can allow specific labeling of sulfydryl structures in polypeptides, carbohydrates, drug molecules, and proteins, as well as in proteins and other biomacromolecules, exhibiting good anti-SARS-CoV-2 activity; and a bioconjugate with trastuzumab according to the present invention can effectively image HER² receptors on the cell surface and show intense fluorescence, and is applicable to the preparation of an imaging reagent for the HER² receptors on the cell surface.

### TECHNICAL FIELD

The present invention relates to synthesis method and application of benzothiaselenazole-1-oxide compound and derivative thereof.

### RELATED ART

Selenium (Se) is a trace element essential for human metabolism, and selenium deficiency leads to the increased incidence of diseases such as cancer, cardiovascular disease, and Kaschin-Beck disease. Also, selenium is one of the most important elements in proteins, and there are more than 25 types of specific selenoproteins in animals, in particular vertebrates. Due to the unique nucleophilic and electrophilic characteristics of selenium atoms, several selenoproteins, including glutathione peroxidases (GPxs) and thioredoxin reductases (TrxRs), are crucial for maintaining various physiological functions in the body. They continuously resist the production of reactive oxygen species (ROS) primarily through a reversible oxidation-reduction process to thereby achieve equilibrium between oxidation and reduction, providing the basis for targeted therapy of diseases induced by aberrant ROS. In light of the importance of selenium, people with selenium deficiency need additional selenium supplementation. Selenium supplement compounds in an early stage are mostly inorganic with high toxicity, which reduces their value. Organic selenium compounds have attracted extensive attention due to their low toxicity and strong pharmacological activity. At present, organic selenium compounds have been developed as more effective and highly selective therapeutic agents for the treatment of many diseases caused by the corresponding abnormal expression of selenoproteins, among which selenium-containing show special significance. Benzoselenazodone has been studied most, because it can exert potent anti-inflammatory, anticancer, antimicrobial, antiviral, and neuroprotective effects as GPx and TrxR mimics by biochemically reacting with thiol groups of cysteines (Cys) on proteins to form Se—S bonds, for example, SARS-CoV-2 M^{pro} proteins. In addition, the organic selenium compounds have been widely used as fluorescent probes and in the construction of DNA compounds, which further highlight their value.

In view of the important effects of organic selenium compounds mentioned above, the academic community has been committed to developing effective synthesis methods. Conventional cross-coupling methods can be used for the synthesis of organic selenium compounds, but they are based on the pre-functionalization of substrates and therefore have some inherent shortcomings, such as harsh reaction conditions, low atom economy, poor substrate/functional group tolerance, and cumbersome preparation of starting materials. To overcome these shortcomings, a transition metal-catalyzed C—H functionalization strategy has been used to synthesize the organic selenium compounds. However, pre-activated selenium sources (for example, dichloroselenide and diselenide) are often involved, which inevitably lead to low atomic economy and complex processing. From the perspective of green and sustainable chemistry, it is ideal to construct organic selenium compounds through direct C—H functionalization using elemental selenium as a selenide reagent. So far, Nishihara and Miura have reported three pioneering solutions, respectively. Miura reported the direct construction of isoselenazole cyclic compounds by the rhodium-catalyzed oxidative cyclization of phenylimine and elemental selenium, but the product structure was relatively simple. Nishihara reported the construction of benzoisoselenazodone compounds by the rhodium-catalyzed cyclic selenization of benzamide and elemental selenium, in which 8-aminoquinoline bidentate directing groups were required, which greatly limited the scope of substrate application. Therefore, there is an urgent need to develop an efficient and universal synthesis method for synthesizing novel organic selenium compounds, with mild conditions and convenient operation.

### SUMMARY OF INVENTION

An object of the present invention is to provide a benzothiaselenazole-1-oxidebenzothiaselenazole-1-oxide compound or a derivative thereof, and synthesis methods therefor and applications thereof.

The present invention is implemented by the following technical solution:

A benzothiaselenazole-1-oxide compound of formula I:

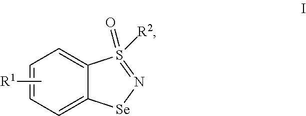

wherein R¹ is selected from any one of the following groups: H, C1-C4 alkyl, halogen, phenyl, —CF₃, —OCF₃, —NO₂, —CO₂Me, —OTf, —OTs or —OMe; and R² is selected from any one of the following groups: halogen, C1-C3 alkyl, cycloalkyl, chloroalkyl or phenyl or substituted phenyl or naphthyl. Preferably, the chloroalkyl is C1-C4 chloroalkyl.

Preferably, the benzothiaselenazole-1-oxide compound of formula I is selected from the following compounds:

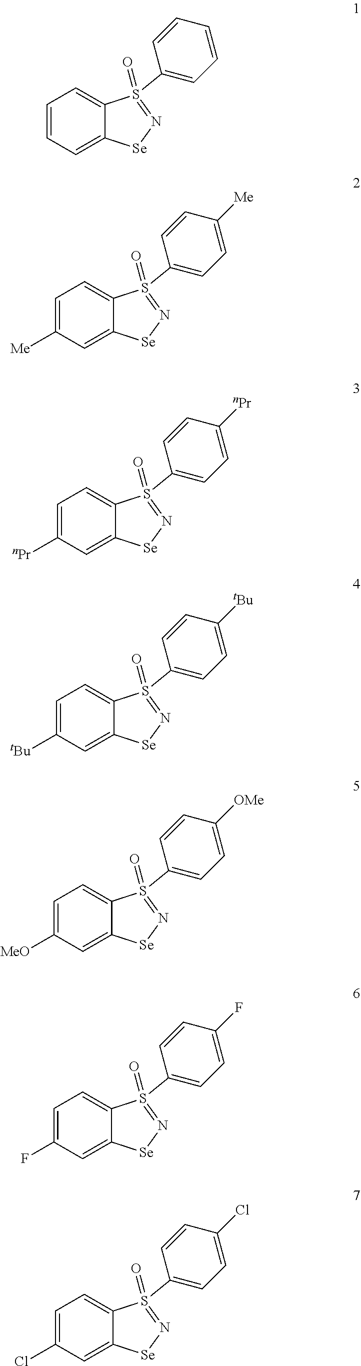

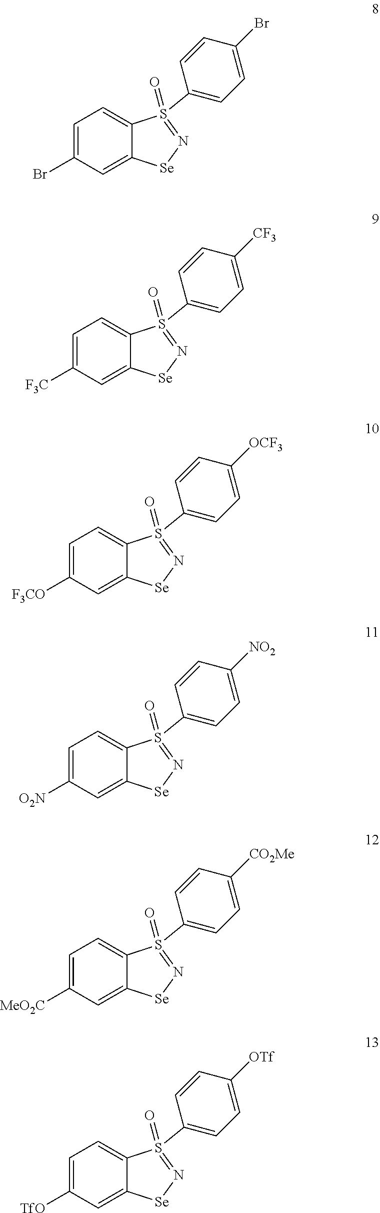 [SMILES: O=S1(c2ccc(Br)cc2)=N[Se]c2cc(Br)ccc21 ; O=S1(c2ccc(C(F)(F)F)cc2)=N[Se]c2cc(C(F)(F)F)ccc21 ; O=S1(c2ccc(OC(F)(F)F)cc2)=N[Se]c2cc(OC(F)(F)F)ccc21 ; O=[N+]([O-])c1ccc(S2(=O)=N[Se]c3cc([N+](=O)[O-])ccc32)cc1 ; COC(=O)c1ccc(S2(=O)=N[Se]c3cc(C(=O)OC)ccc32)cc1 ; O=S(=O)(Oc1ccc(S2(=O)=N[Se]c3cc(OS(=O)(=O)C(F)(F)F)ccc32)cc1)C(F)(F)F]

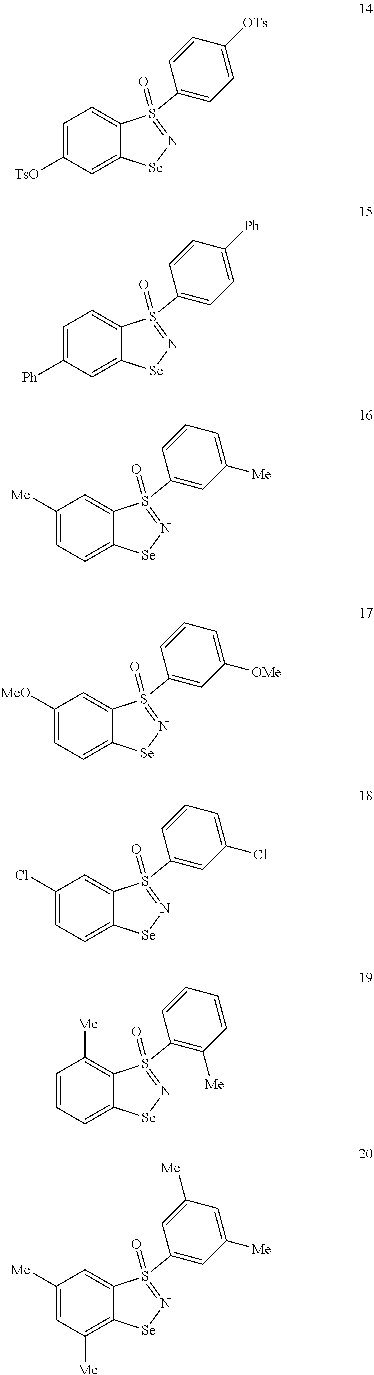

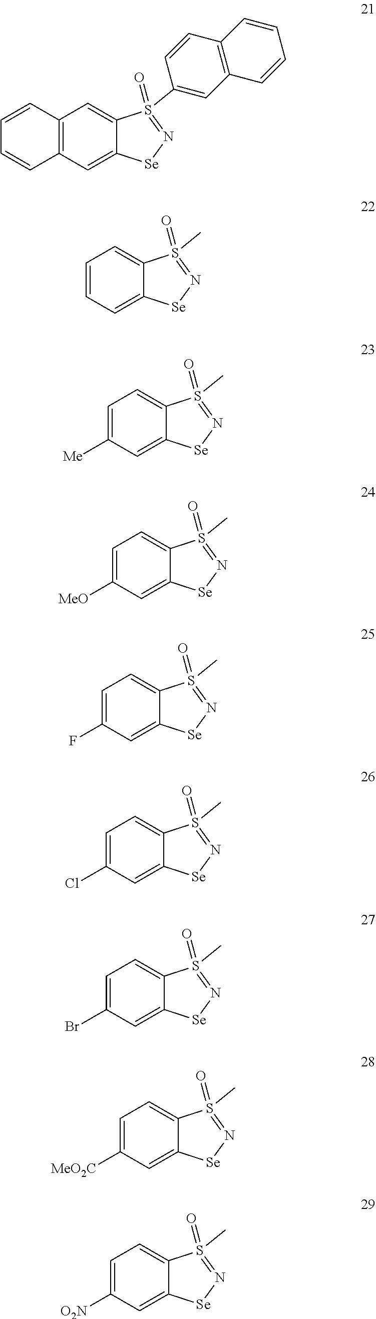

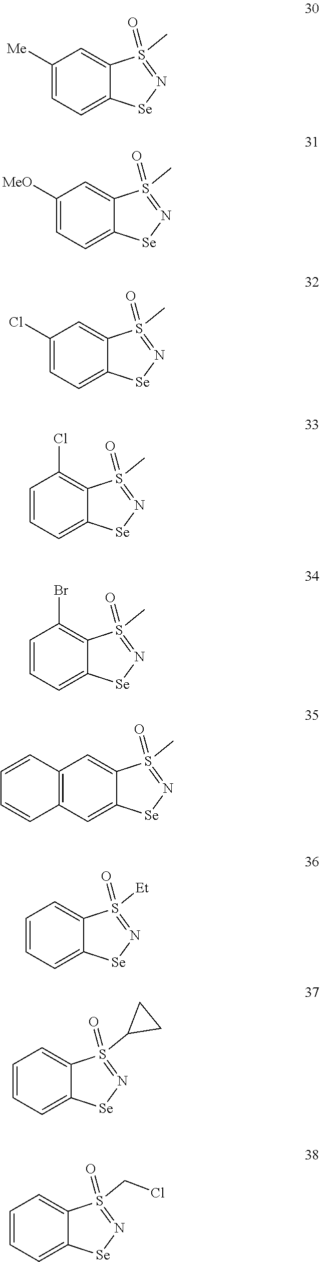

A synthesis method for a benzothiaselenazole-1-oxide compound of formula I includes the step of: reacting selenium with sulfoximine of formula III, which are taken as starting materials, under catalysis of rhodium at 90-110° C. to obtain the benzothiaselenazole-1-oxide compound:

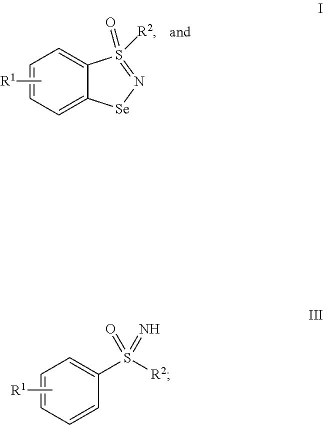

wherein R¹ is selected from any one of the following groups: H, C1-C4 alkyl, halogen, phenyl, —CF₃, —OCF₃, —NO₂, —CO₂Me, —OTf, —OTs or —OMe; and R² is selected from any one of the following groups: halogen, C1-C3 alkyl, cycloalkyl, chloroalkyl or phenyl or substituted phenyl or naphthyl.

Preferably, the chloroalkyl is C1-C4 chloroalkyl.

When the reaction temperature is 100° C., reaction equations are as follows:

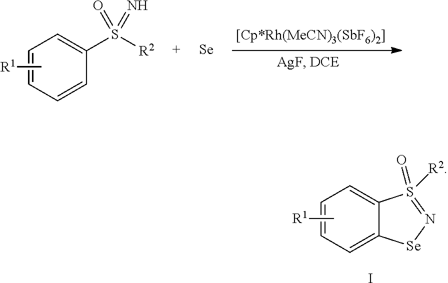

The synthesis method in particular includes the steps of: adding [Cp*Rh(MeCN)₃(SbF₂)₂], AgF, selenium, and sulfoximine into a sealed tube to allow for dissolution in dichloroethane, and letting react in an oil bath at 100° C., wherein a molar ratio of AgF to selenium to sulfoximine is 2.5:(3-4):1.

The present invention further provides a synthesis method for an enantiomer of benzothiaselenazole-1-oxide compound of formula II. The method includes the step of: subjecting selenium and sulfoximine of formula IV, which are taken as starting materials, to a direct C—H functionalization reaction by a chiral phosphoric acid ligand at 55-65° C. to obtain the enantiomer of benzothiaselenazole-1-oxide compound;

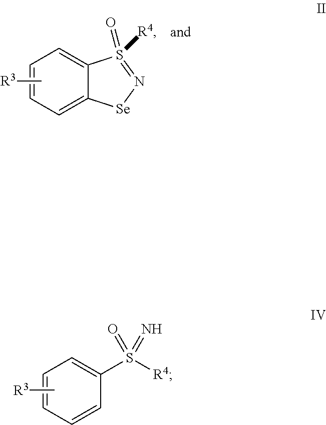

wherein R³ is selected from any one of the following groups: H, C1-C4 alkyl, halogen, phenyl, —CF₃, —OCF₃, —NO₂, —CO₂Me, —OTf, —OTs or —OMe; and R⁴ is selected from any one of the following groups: phenyl or substituted phenyl or naphthyl.

When the reaction temperature is 60° C., reaction equations are as follows:

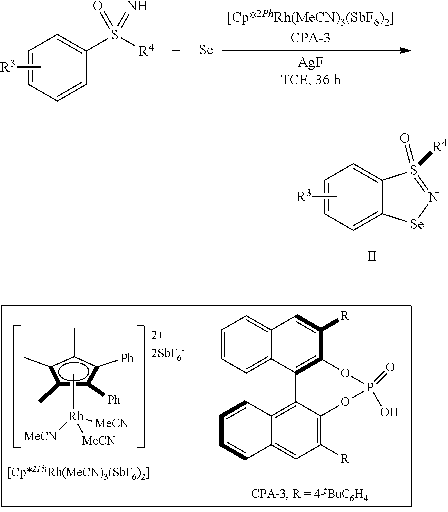

The synthesis method in particular includes the steps of: adding [Cp*^{2ph}Rh(MeCN)₃(SbF₆)₂], AgF, selenium, sulfoximine, and the chiral phosphoric acid ligand (CPA-3) into a sealed tube to allow for dissolution in trichloroethylene, and letting react in an oil bath at 60° C. to obtain the enantiomer of benzothiaselenazole-1-oxide compound, wherein a molar ratio of AgF to selenium to sulfoximine is 2.5:(3-4):1.

Preferably, the enantiomer of benzothiaselenazole-1-oxide compound of formula II is selected from the following compounds:

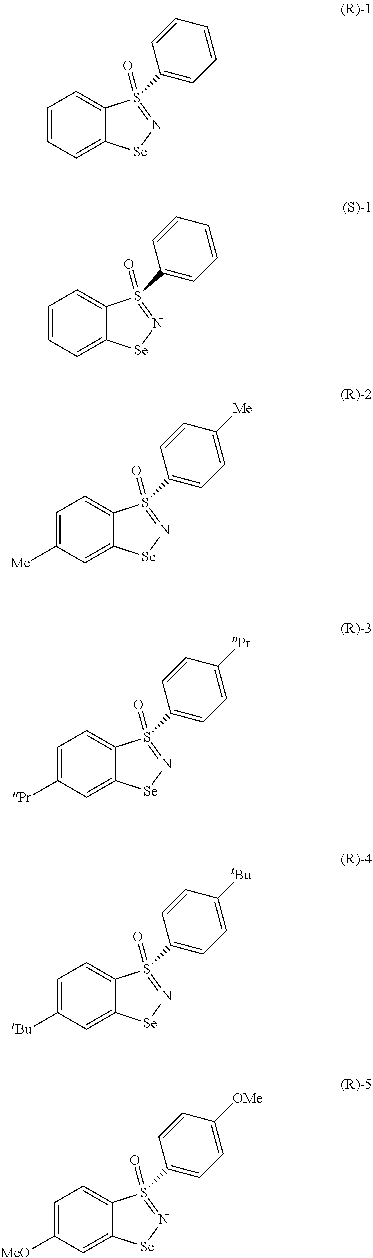

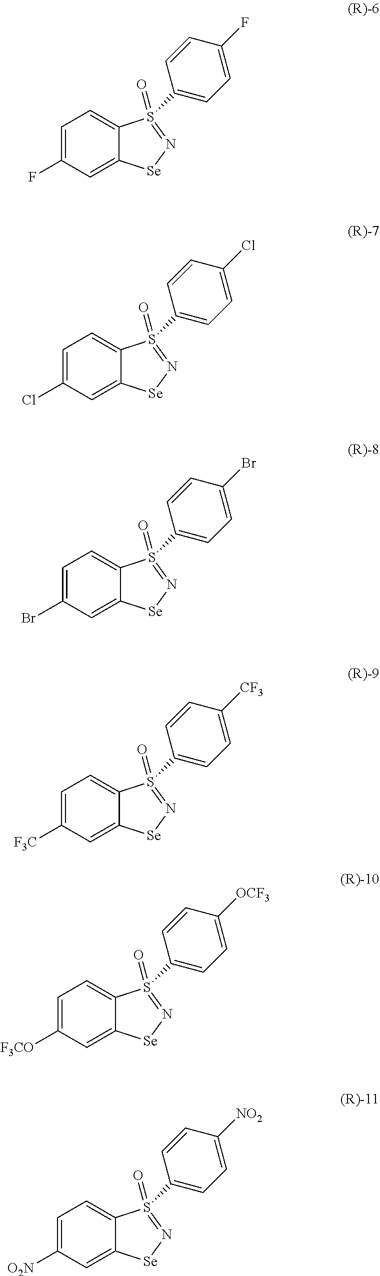 [SMILES: O=[S@@]1(c2ccc(F)cc2)=N[Se]c2cc(F)ccc21 ; O=[S@@]1(c2ccc(Cl)cc2)=N[Se]c2cc(Cl)ccc21 ; O=[S@@]1(c2ccc(Br)cc2)=N[Se]c2cc(Br)ccc21 ; O=[S@@]1(c2ccc(C(F)(F)F)cc2)=N[Se]c2cc(C(F)(F)F)ccc21 ; O=[S@@]1(c2ccc(OC(F)(F)F)cc2)=N[Se]c2cc(OC(F)(F)F)ccc21 ; O=[N+]([O-])c1ccc([S@]2(=O)=N[Se]c3cc([N+](=O)[O-])ccc32)cc1]

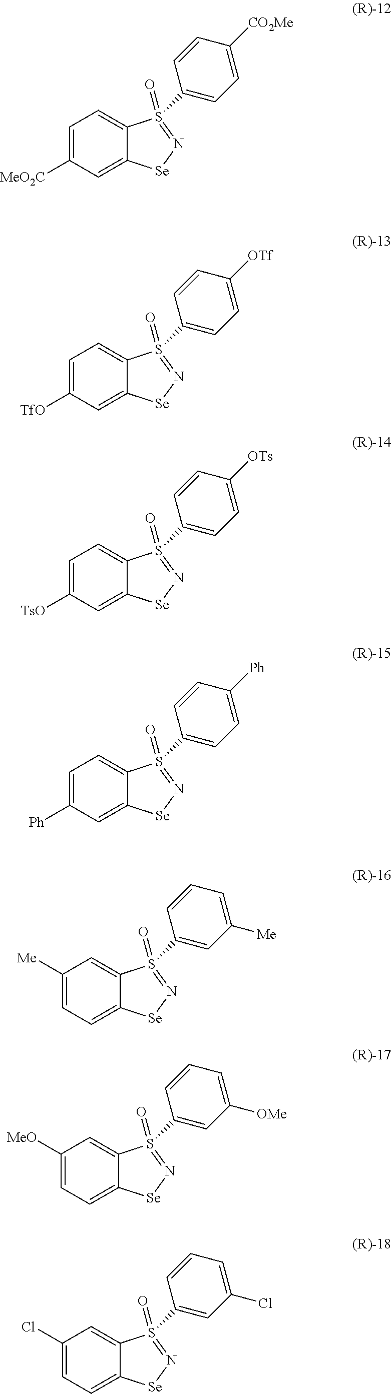

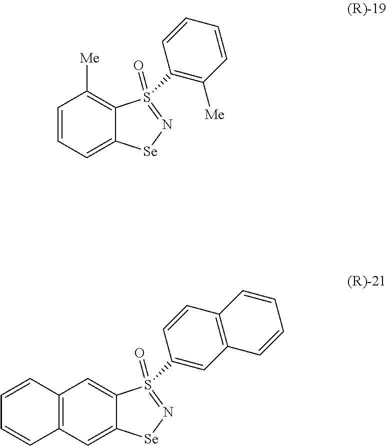 [SMILES: Cc1ccccc1[S@]1(=O)=N[Se]c2cccc(C)c21 ; O=[S@@]1(c2ccc3ccccc3c2)=N[Se]c2cc3ccccc3cc21]

The benzothiaselenazole-1-oxide compound of formula I or the enantiomer of benzothiaselenazole-1-oxide compound of formula II according to the present invention exhibits good anti-SARS-CoV-2 activity, which is manifested as: excellent inhibitory activity in SARS-CoV-2 virus-infected primate Vero cells, and the effective inhibition rate of greater than 90% at a concentration of 10 μM; and moreover, superior inhibitory activity of the compound 1 and its enantiomer against the M^{pro} protein of SARS-CoV-2 as compared to an ebselen (EBS) control group. Further studies have shown that the benzothiaselenazole-1-oxide compound provided by the present invention exerts activity by covalently binding to the key Cys145 site in the M^{pro} protein of the SARS-CoV-2.

Therefore, the present invention further claims application of the benzothiaselenazole-1-oxide compound of formula I or the enantiomer of benzothiaselenazole-1-oxide compound of formula II in preparation of an anti-SARS-CoV-2 medicament.

The present invention further claims application of the benzothiaselenazole-1-oxide compound of formula I according to claim 1 or the enantiomer of benzothiaselenazole-1-oxide compound of formula II according to claim 4 in modification of sulfydryl-containing molecules based on Se—S covalent linkage, wherein the sulfydryl-containing molecules include amino acids, polypeptide and derivatives thereof, carbohydrates, and other pharmaceutical molecules.

With a benzothiaselenazole-1-oxide derivative compound 12 provided by the present invention as a starting material, a bifunctional selenium linker compound L1 is synthesized through processes such as hydrolysis and amide condensation, and this compound can be further subjected to Se—S bond linkage with trastuzumab to obtain a bioconjugate C1, which is then linked with an Alexa Fluor®555 DIBO dye by means of a “click” reaction of azide and alkyne to obtain a trastuzumab-Alexa Fluor®555 conjugate C2. Fluorescence microscopy images clearly show that C2 can efficiently image an HER² receptor on a cell surface, and compared to negative MCF-7 (HER²⁻) cells, BT474 (HER²⁺) cells with high expression of HER² show intense fluorescence.

With the compound 12 as the starting material, the bifunctional selenium linker compound L1 is synthesized by hydrolysis and amide condensation, with reaction equations as follows:

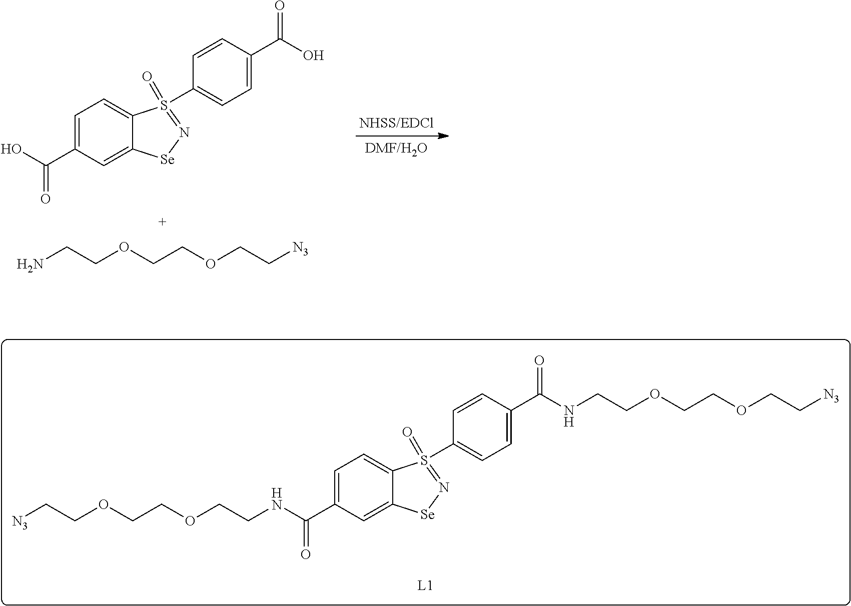 [SMILES: O=C(O)c1ccc(S2(=O)=N[Se]c3cc(C(=O)O)ccc32)cc1 ; [N-]=[N+]=NCCOCCOCCN ; [N-]=[N+]=NCCOCCOCCNC(=O)c1ccc(S2(=O)=N[Se]c3cc(C(=O)NCCOCCOCCN=[N+]=[N-])ccc32)cc1]

The process of labeling the trastuzumab with the bifunctional selenium linker compound L1 is represented as follows:

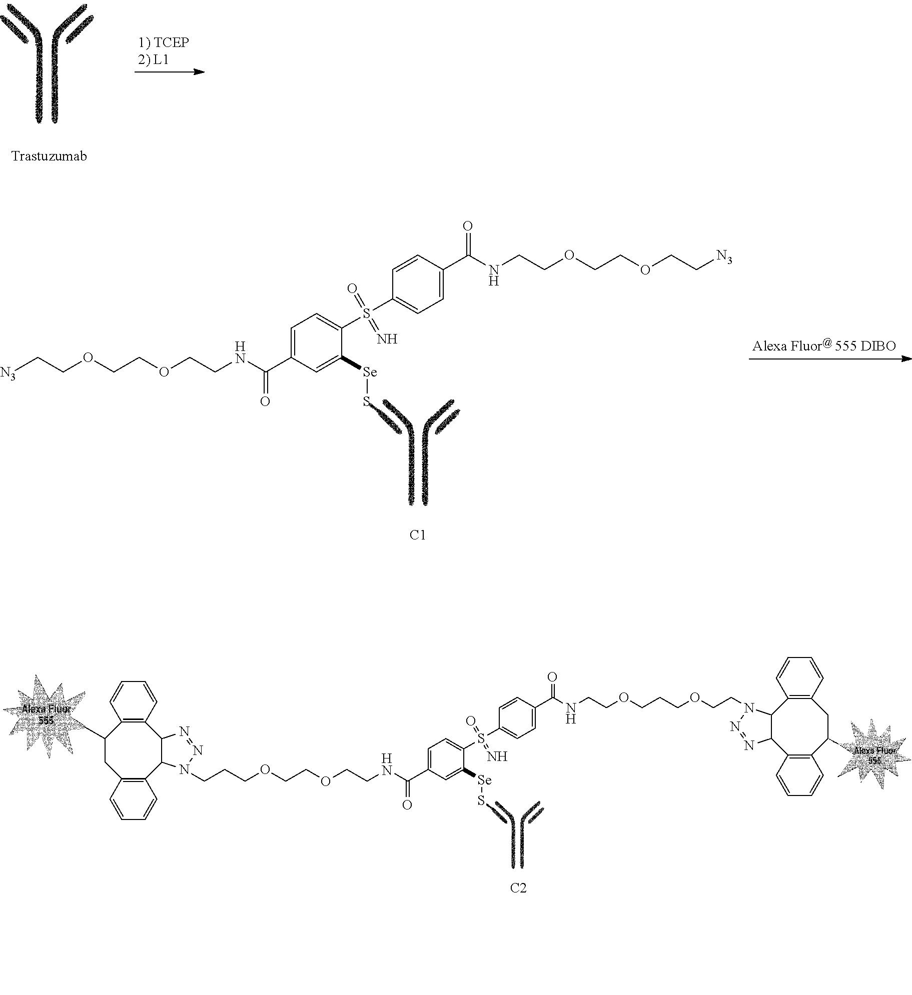

Therefore, the present invention further claims application of a trastuzumab bioconjugate of the benzothiaselenazole-1-oxide compound of formula I or the enantiomer of benzothiaselenazole-1-oxide compound of formula II in preparation of an imaging reagent for an HER² receptor on a cell surface.

The present invention further claims application of the benzothiaselenazole-1-oxide compound of formula I or the enantiomer of benzothiaselenazole-1-oxide compound of formula II or their derivatives as a selenizing reagent, wherein a novel indole-selenium compound based on the benzothiaselenazole-1-oxide compound is obtained through reaction by taking indole or its derivative as a reactant and tris(pentafluorophenyl) borane as a catalyst.

In the case of the benzothiaselenazole-1-oxide compound of formula I or the enantiomer of benzothiaselenazole-1-oxide compound of formula II, reaction equations are as follows:

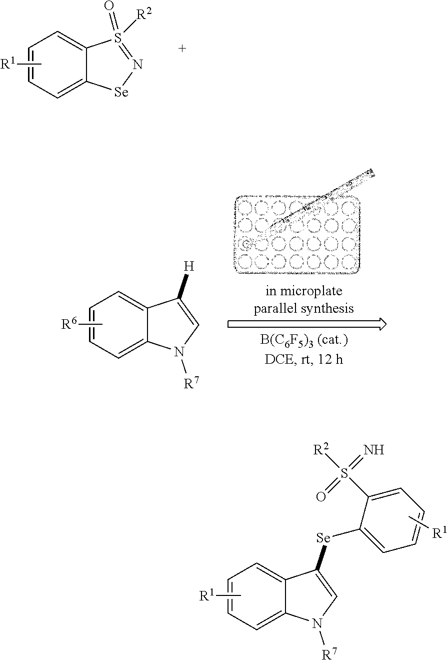

A derivative of the benzothiaselenazole-1-oxide compound of formula I is selected from the following compounds:

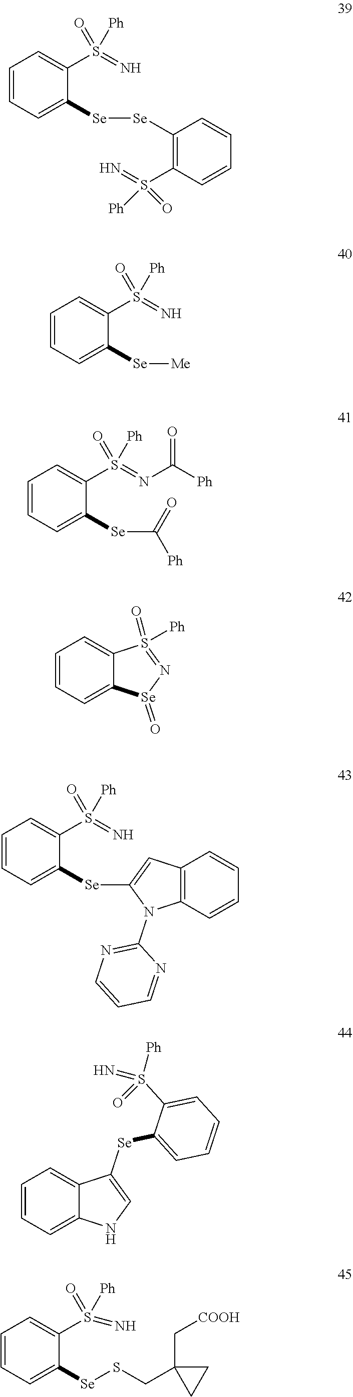

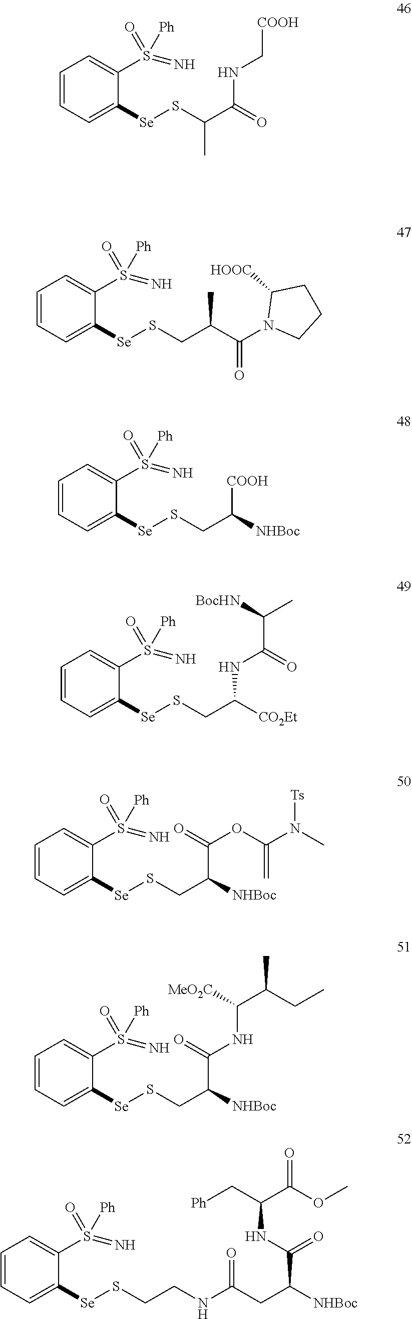

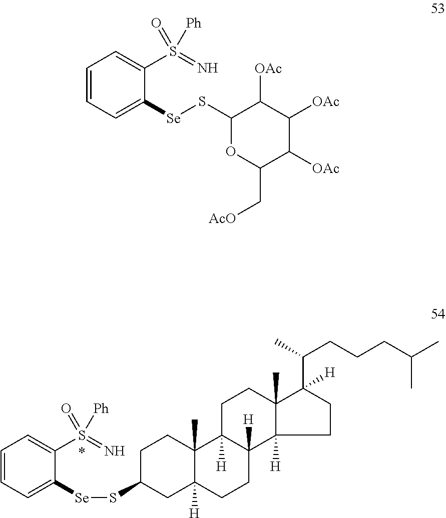

In particular, an indole derivative is DNA-labeled indole.

Reaction equations are as follows:

The present invention further claims a novel organic selenium compound of formula V based on a benzothiaselenazole-1-oxide compound:

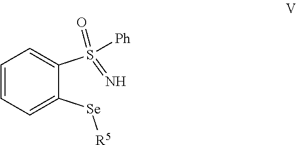

wherein R⁵ is selected from any one of the following groups: alkyl, acyl, substituted indyl, substituted seleno, or a sulfydryl compound.

The alkyl is preferably C1-C100 alkyl, more preferably C1-C10 alkyl.

In particular, the substituted indyl comprises indole N, and an indole substrate with different substitutions at C2-C7 positions, and substituents comprise alkyl, alkoxy, ester, alkynyl, halogen, nitro and other functional groups, as well as an indole compound labeled with DNA tags at different sites.

The alkyl is preferably C1-C100 alkyl, more preferably C1-C10 alkyl.

The present invention has the following beneficial effects.

1) The present invention provides a novel organic selenium compound (benzothiaselenazole-1-oxide compound) or derivatives thereof and synthesis methods therefor; with sulfoximine and elemental selenium as starting materials, a series of benzothiaselenazole-1-oxide compounds has been synthesized through rhodium-catalyzed direct C—H functionalization reaction; and the advantages of strong compatibility of functional groups, general applicability of substrates, mild conditions, simple operation, high efficiency, and universality are achieved.

2) With sulfoximine and elemental selenium as starting materials, the present invention synthesizes a chiral benzothiaselenazole-1-oxide compound through direct C—H functionalization reaction by means of a chiral phosphoric acid ligand. So far, it have not been reported the construction of chiral organic selenium compounds through direct C—H functionalization using elemental selenium as a selenide reagent.

3) The benzothiaselenazole-1-oxide compound or the derivatives thereof as prepared by the present invention have good application prospects, and can allow specific labeling of sulfydryl groups by Se—S bonds, and show good anti-SARS-CoV-2 activity, and its bioconjugate with trastuzumab can effectively image the HER² receptor on the cell surface and show intense fluorescence, and is applicable to the preparation of an imaging reagent for the HER² receptor on the cell surface.

### BRIEF DESCRIPTION OF DRAWINGS

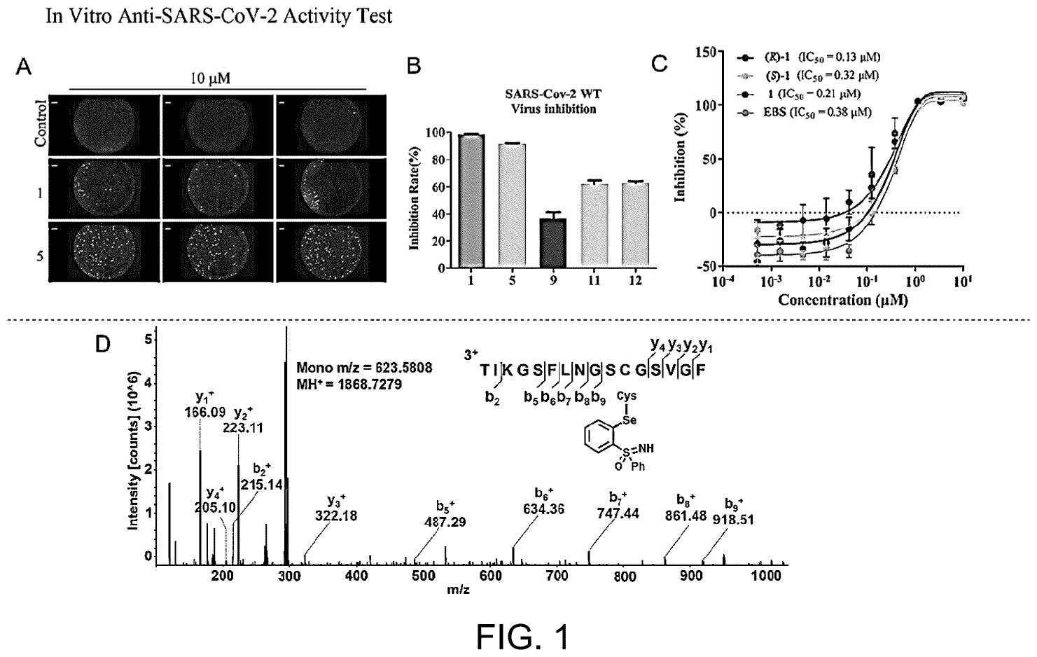

FIG. 1 shows the evaluation of in vitro antiviral activity of Embodiment 77,
wherein, a: the antiviral activity of the compound against SARS-CoV-2 infected primate Vero cells; b: the inhibition rate of different compounds against SARS-CoV-2; c: the half-maximal inhibitory concentration of representative compounds; and d: the MS-MS analysis results of compound 1-labeled M^{pro} proteins.

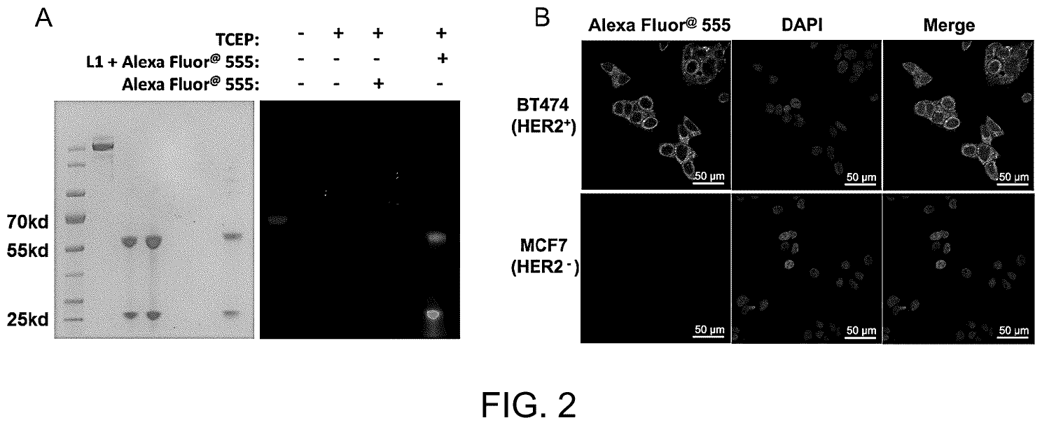

FIG. 2 shows the bio-orthogonal labeling of trastuzumab and the imaging of HER² receptors on the cell surface in Embodiment 78,
wherein, a: the SDS-PAGE analysis of labeled proteins; and b: the imaging of HER² receptors on the cell surface.

### DESCRIPTION OF EMBODIMENTS

The following is intended to further illustrate the present invention, rather than limiting the present invention.

### Embodiment 1: Synthesis of 1-phenylbenzo[d][1,3,2]thiaselenazole-1-ketone (Compound 1)

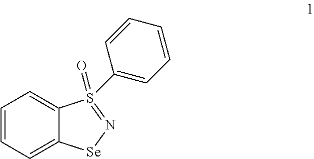 [SMILES: O=S1(c2ccccc2)=N[Se]c2ccccc21]

[Cp*Rh(MeCN)₃(SbF₆)₂] (5 mol %), AgF (0.25 mmol, 2.5 equiv.), selenium (0.3 mmol, 3 equiv.), and sulfoximine (0.1 mmol, 1 equiv.) were added into a 10 mL sealed tube, dissolved in dichloroethane (0.5 ml), and then let react for 10 h in an oil bath at 100° C. After the reaction was completed, EA was added into the reaction tube for dilution; a reaction solution was transferred to a round-bottomed flask and concentrated to obtain a crude product, which was then separated by column chromatography and eluted with an eluent of PE:EA=3:1 to obtain 23.4 mg of a product, with the yield of 81%. ¹H NMR (400 MHZ, CDCl₃): δ 7.99 (d, J=7.6 Hz, 2H), 7.66 (m, 2H), 7.57 (t, J=7.6 Hz, 2H), 7.50 (t, J=7.5 Hz, 1H), 7.40 (d, J=7.9 Hz, 1H), 7.30 (t, J=7.5 Hz, 1H). ¹³C NMR (101 MHz, CDCl₃): δ 141.4, 140.6, 134.7, 133.9, 131.6, 129.8, 129.4, 126.4, 125.7, 124.2.

### Embodiment 2: Synthesis of 5-methyl-1-(p-methylphenyl)benzo[d][1,3,2]thiaselenazole-1-ketone (Compound 2)

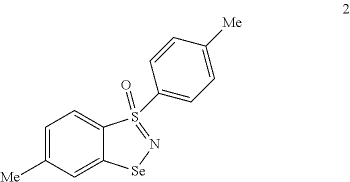 [SMILES: Cc1ccc(S2(=O)=N[Se]c3cc(C)ccc32)cc1]

A reference was made to Embodiment 1 for the preparation method, with the difference that 21.0 mg of the target compound was obtained using iminodi-p-methylphenyl-λ⁶-sulfone as a substrate, with the yield of 65%, ¹H NMR (400 MHZ, CDCl₃): δ 7.85 (d, J=8.2 Hz, 2H), 7.41 (s, 1H), 7.34 (d, J=8.1 Hz, 2H), 7.28-7.24 (m, 1H), 7.08 (d, J=8.1 Hz, 1H), 2.44 (s, 3H), 2.41 (s, 3H). ¹³C NMR (101 MHz, CDCl₃): δ 144.9, 142.6, 141.7, 137.8, 132.7, 130.0, 129.7, 127.8, 125.2, 124.0, 21.8, 21.7.

### Embodiment 3: Synthesis of 5-propyl-1-(p-propylphenyl)benzo[d][1,3,2]thiaselenazole-1-ketone (Compound 3)

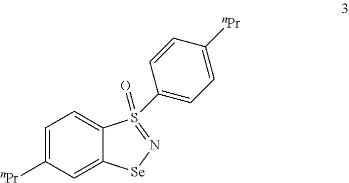

A reference was made to Embodiment 1 for the preparation method, with the difference that 24.3 mg of the target compound was obtained using iminodi-p-propylphenyl-λ⁶-sulfone as a substrate, with the yield of 64%. ¹H NMR (400 MHZ, CDCl₃): δ 7.86 (d, J=8.1 Hz, 2H), 7.40 (s, 1H), 7.33 (d, J=8.1 Hz, 2H), 7.28 (d, J=8.2 Hz, 1H), 7.08 (d, J=8.1 Hz, 1H), 2.64 (q, J=8.4 Hz, 4H), 1.68-1.60 (m, 4H), 0.92 (t, J=7.2 Hz, 6H). ¹³C NMR (101 MHz, CDCl₃): δ 149.6, 147.3, 141.6, 137.8, 132.8, 129.8, 129.4, 127.3, 125.3, 123.5, 38.0, 38.0, 24.4, 24.3, 13.9.

### Embodiment 4: Synthesis of 5-tert-butyl-1-(p-phenyl)benzo[d][1,3,2]thiaselenazole-1-ketone (Compound 4)

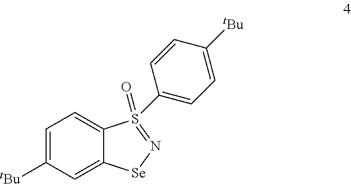 [SMILES: CC(C)(C)c1ccc(S2(=O)=N[Se]c3cc(C(C)(C)C)ccc32)cc1]

A reference was made to Embodiment 1 for the preparation method, with the difference that 17.0 mg of the target compound was obtained using iminodi-p-tert-butylphenyl-λ⁶-sulfone as a substrate, with the yield of 42%. ¹H NMR (400 MHZ, CDCl₃): δ 7.89 (d, J=8.5 Hz, 2H), 7.61 (s, 1H), 7.55 (d, J=8.5 Hz, 2H), 7.34 (m, 2H), 1.34 (m, 18H). ¹³C NMR (101 MHz, CDCl₃): δ 157.8, 155.7, 141.7, 137.7, 132.4, 129.5, 126.4, 125.2, 124.7, 120.4, 31.3, 31.2.

### Embodiment 5: Synthesis of 5-methoxy-1-(4-methoxyphenyl)benzo[d][1,3,2]thiaselenazole-1-ketone (Compound 5)

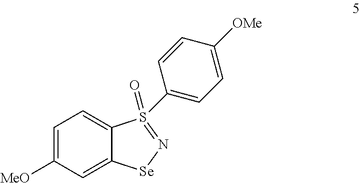 [SMILES: COc1ccc(S2(=O)=N[Se]c3cc(OC)ccc32)cc1]

A reference was made to Embodiment 1 for the preparation method, with the difference that 25.3 mg of the target compound was obtained using iminodi-p-methoxyphenyl-λ⁶-sulfone as a substrate, with the yield of 71%. ¹H NMR (400 MHZ, CDCl₃): δ 7.89 (d, J=8.8 Hz, 2H), 7.25 (d, J=8.8 Hz, 1H), 7.02 (m, 3H), 6.81 (m, J=8.8 Hz, 1H), 3.87 (s, 3H), 3.84 (s, 3H). ¹³C NMR (101 MHZ, CDCl₃): δ 163.9, 162.5, 143.9, 132.2, 131.7, 127.9, 126.5, 115.1, 114.5, 106.7, 56.0, 55.9.

### Embodiment 6: Synthesis of 5-fluoro-1-(4-fluorophenyl)benzo[d][1,3,2]thiaselenazole-1-ketone (Compound 6)

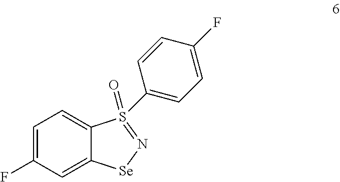 [SMILES: O=S1(c2ccc(F)cc2)=N[Se]c2cc(F)ccc21]

A reference was made to Embodiment 1 for the preparation method, with the difference that 29.7 mg of the target compound was obtained using iminodi-p-fluorophenyl-λ⁶-sulfone as a substrate, with the yield of 90%. ¹H NMR (400 MHZ, CDCl₃): δ 8.00 (dd, J=8.7, 5.1 Hz, 2H), 7.33 (m, 2H), 7.25 (t, J=8.4 Hz, 2H), 7.05-6.99 (m, 1H). ¹³C NMR (101 MHz, CDCl₃): δ 166.3 (d, J=259.6 Hz), 164.7 (d, J=257.6 Hz), 144.6 (d, J=9.5 Hz), 136.4, 132.6 (d, J=9.8 Hz), 130.9, 127.4 (d, J=10.4 Hz), 116.8 (d, J=22.8 Hz), 115.2 (d, J=25.0 Hz), 110.9 (d, J=25.7 Hz). ¹⁹F NMR (376 MHz, CDCl₃): δ −103.1, −106.9.

### Embodiment 7: Synthesis of 5-chloro-1-(4-chlorophenyl)benzo[d][1,3,2]thiaselenazole-1-ketone (Compound 7)

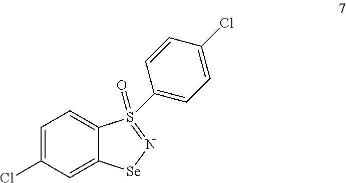 [SMILES: O=S1(c2ccc(Cl)cc2)=N[Se]c2cc(Cl)ccc21]

A reference was made to Embodiment 1 for the preparation method, with the difference that 33.3 mg of the target compound was obtained using iminodi-p-chlorophenyl-λ⁶-sulfone as a substrate, with the yield of 92%. ¹H NMR (400 MHZ, CDCl₃): δ 7.90 (d, J=8.6 Hz, 2H), 7.63 (s, 1H), 7.54 (d, J=8.6 Hz, 2H), 7.31-7.25 (m, 2H). ¹³C NMR (101 MHz, CDCl₃): δ 143.7, 141.2, 139.0, 138.8, 132.9, 131.1, 129.8, 127.2, 126.4, 124.0.

### Embodiment 8: Synthesis of 5-bromo-1-(4-bromophenyl)benzo[d][1,3,2]thiaselenazole-1-ketone (Compound 8)

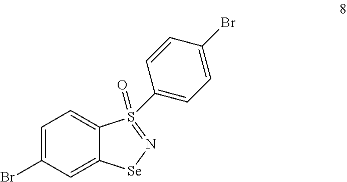 [SMILES: O=S1(c2ccc(Br)cc2)=N[Se]c2cc(Br)ccc21]

A reference was made to Embodiment 1 for the preparation method, with the difference that 43.1 mg of the target compound was obtained using iminodi-p-bromophenyl-λ⁶-sulfone as a substrate, with the yield of 96%. ¹H NMR (400 MHZ, CDCl₃): δ 7.90 (d, J=8.6 Hz, 2H), 7.63 (s, 1H), 7.54 (d, J=8.6 Hz, 2H), 7.28 (d, J=2.3 Hz, 2H). ¹³C NMR (101 MHz, CDCl₃): δ 144.0, 139.4, 133.3, 132.8, 131.2, 130.0, 129.9, 127.4, 126.9, 126.6.

### Embodiment 9: Synthesis of 5-trifluoromethyl-1-(4-trifluoromethylphenyl)benzo[d][1,3,2]thiaselenazole-1-ketone (Compound 9)

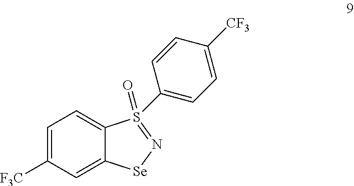 [SMILES: O=S1(c2ccc(C(F)(F)F)cc2)=N[Se]c2cc(C(F)(F)F)ccc21]

A reference was made to Embodiment 1 for the preparation method, with the difference that 31.1 mg of the target compound was obtained using iminodi-p-trifluoromethylphenyl-λ⁶-sulfone as a substrate, with the yield of 72%. ¹H NMR (400 MHZ, CDCl₃): δ 8.13 (d, J=8.2 Hz, 2H), 7.97 (s, 1H), 7.86 (d, J=8.3 Hz, 2H), 7.57 (d, J=8.4 Hz, 1H), 7.51 (d, J=8.3 Hz, 1H). ¹³C NMR (101 MHz, CDCl₃): δ 143.6, 143.2, 136.3, 136.1 (q, J=33.2 Hz), 134.0 (q, J=33.0 Hz), 130.5, 126.7 (d, J=3.4 Hz), 126.4, 124.6, 123.9 (d, J=3.1 Hz), 121.8 (d, J=4.1 Hz), 119.1. ¹⁹F NMR (376 MHz, CDCl₃): δ −62.7, −63.1.

### Embodiment 10: Synthesis of 5-trifluoromethoxy-1-(4-trifluoromethoxyphenyl)benzo[d][1,3,2]thiaselenazole-1-ketone (Compound 10)

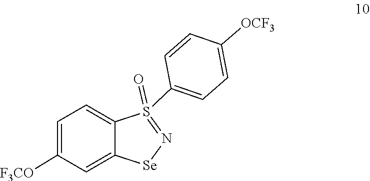 [SMILES: O=S1(c2ccc(OC(F)(F)F)cc2)=N[Se]c2cc(OC(F)(F)F)ccc21]

A reference was made to Embodiment 1 for the preparation method, with the difference that 25.4 mg of the target compound was obtained using iminodi-p-trifluoromethoxyphenyl-λ⁶-sulfone as a substrate, with the yield of 55%. ¹H NMR (400 MHZ, CDCl₃): δ 8.05 (d, J=8.6 Hz, 2H), 7.49 (s, 1H), 7.45-7.38 (m, 3H), 7.17 (d, J=8.5 Hz, 1H). ¹³C NMR (101 MHZ, CDCl₃): δ 153.5, 151.7, 144.4, 138.2, 132.3, 132.1, 127.1, 121.6 (d, J=2.4 Hz), 121.1, 119.6, 119.0 (d, J=2.4 Hz), 115.9.

¹⁹F NMR (376 MHZ, CDCl₃): δ −57.6.

### Embodiment 11: Synthesis of 5-nitro-1-(4-nitrophenyl)benzo[d][1,3,2]thiaselenazole-1-ketone (Compound 11)

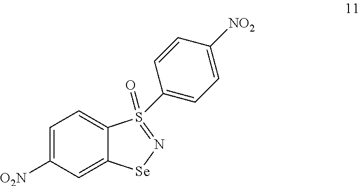 [SMILES: O=[N+]([O-])c1ccc(S2(=O)=N[Se]c3cc([N+](=O)[O-])ccc32)cc1]

A reference was made to Embodiment 1 for the preparation method, with the difference that 23.9 mg of the target compound was obtained using iminodi-p-nitrophenyl-λ⁶-sulfone as a substrate, with the yield of 62%. ¹H NMR (400 MHZ, DMSO-d₆): δ 8.99 (s, 1H), 8.46 (d, J=8.7 Hz, 2H), 8.15 (dd, J=11.9, 5.0 Hz, 3H), 7.85 (d, J=8.7 Hz, 1H). ¹³C NMR (101 MHz, DMSO-d₆): δ 150.7, 149.3, 145.3, 144.7, 136.7, 130.7, 126.8, 124.9, 122.2, 121.9.

### Embodiment 12: Synthesis of 5-methylformyl-1-(4-methylformylphenyl)benzo[d][1,3,2]thiaselenazole-1-ketone (Compound 12)

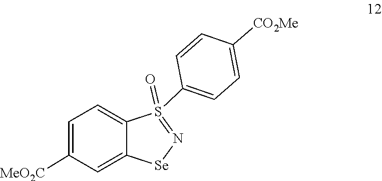 [SMILES: COC(=O)c1ccc(S2(=O)=N[Se]c3cc(C(=O)OC)ccc32)cc1]

A reference was made to Embodiment 1 for the preparation method, with the difference that 23.9 mg of the target compound was obtained using iminodi-p-methylformylphenyl-λ⁶-sulfone as a substrate, with the yield of 58%. ¹H NMR (400 MHZ, CDCl₃): δ 8.36 (s, 1H), 8.23 (d, J=8.5 Hz, 2H), 8.06 (d, J=8.4 Hz, 2H), 7.94 (d, J=8.3 Hz, 1H), 7.44 (d, J=8.3 Hz, 1H), 3.97 (m, 6H). ¹³C NMR (101 MHz, CDCl₃): δ 165.6, 165.5, 143.9, 142.4, 136.8, 135.3, 133.3, 130.6, 129.9, 127.8, 125.9, 125.5, 53.0, 52.9.

### Embodiment 13: Synthesis of 5-trifluoromethanesulfonyl-1-(4-trifluoromethanesulfonylphenyl)benzo[d][1,3,2]thiaselenazol-1-ketone (Compound 13)

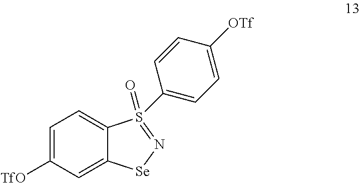 [SMILES: O=S(=O)(Oc1ccc(S2(=O)=N[Se]c3cc(OS(=O)(=O)C(F)(F)F)ccc32)cc1)C(F)(F)F]

A reference was made to Embodiment 1 for the preparation method, with the difference that 40.6 mg of the target compound was obtained using iminodi-p-trifluoromethanesulfonylphenyl-λ⁶-sulfone as a substrate, with the yield of 69%. ¹H NMR (400 MHz, CDCl₃): δ 8.11 (d, J=8.9 Hz, 2H), 7.60 (d, J=1.9 Hz, 1H), 7.51 (d, J=8.8 Hz, 2H), 7.48 (d, J=8.7 Hz, 1H), 7.24 (dd, J=8.7, 1.9 Hz, 1H). ¹³C NMR (101 MHZ, CDCl₃): δ 153.4, 151.6, 145.1, 140.2, 133.4, 132.4, 127.5, 122.7, 120.4, 117.4, 117.2, 117.1. ¹⁹F NMR (376 MHz, CDCl₃): δ −72.40, −72.45.

### Embodiment 14: Synthesis of 5-p-toluenesulfonyloxy-1-(4-p-toluenesulfonyloxyphenyl)benzo[d][1,3,2]thiaselenazole-1-ketone (Compound 14)

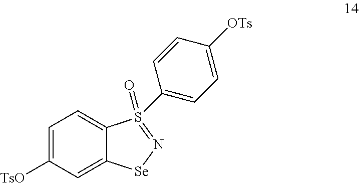 [SMILES: Cc1ccc(S(=O)(=O)Oc2ccc(S3(=O)=N[Se]c4cc(OS(=O)(=O)c5ccc(C)cc5)ccc43)cc2)cc1]

A reference was made to Embodiment 1 for the preparation method, with the difference that 37.7 mg of the target compound was obtained using iminodi-p-toluenesulfonyloxyphenyl-λ⁶-sulfone as a substrate, with the yield of 58%. ¹H NMR (400 MHZ, CDCl₃): δ 7.90 (d, J=8.7 Hz, 2H), 7.74 (t, J=7.2 Hz, 4H), 7.38-7.33 (m, 4H), 7.31 (s, 1H), 7.26 (d, J=1.9 Hz, 1H), 7.21 (d, J=8.7 Hz, 2H), 6.93 (d, J=8.6 Hz, 1H), 2.47 (s, 6H). ¹³C NMR (101 MHz, CDCl₃): δ 153.9, 152.1, 146.3, 144.0, 138.6, 132.5, 131.9, 131.83, 131.76, 130.3, 128.6, 126.8, 123.4, 121.2, 118.0, 21.9.

### Embodiment 15: Synthesis of 5-phenyl-1-biphenylbenzo[d][1,3,2]thiaselenazole-1-ketone (Compound 15)

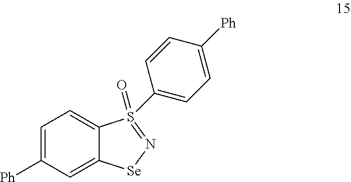 [SMILES: O=S1(c2ccc(-c3ccccc3)cc2)=N[Se]c2cc(-c3ccccc3)ccc21]

A reference was made to Embodiment 1 for the preparation method, with the difference that 30.0 mg of the target compound was obtained using iminodi-biphenyl-λ⁶-sulfone as a substrate, with the yield of 68%. ¹H NMR (400 MHZ, CDCl₃): δ 8.08 (d, J=8.4 Hz, 2H), 7.82-7.76 (m, 3H), 7.62 (d, J=7.2 Hz, 2H), 7.58 (d, J=7.0 Hz, 2H), 7.52-7.41 (m, 8H). ¹³C NMR (101 MHz, CDCl₃): δ 146.9, 145.1, 142.4, 139.3, 139.2, 139.0, 133.5, 130.3, 129.22, 129.21, 128.9, 128.8, 128.0, 127.7, 127.6, 126.1, 125.9, 122.4.

### Embodiment 16: Synthesis of 6-methyl-1-m-methylphenylbenzo[d][1,3,2]thiaselenazole-1-ketone (Compound 16)

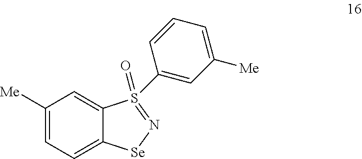 [SMILES: Cc1cccc(S2(=O)=N[Se]c3ccc(C)cc32)c1]

A reference was made to Embodiment 1 for the preparation method, with the difference that 17.0 mg of the target compound was obtained using iminodi-m-methylphenyl-λ⁶-sulfone as a substrate, with the yield of 53%. ¹H NMR (400 MHZ, CDCl₃): δ 7.82 (m, 1H), 7.77 (s, 1H), 7.51 (d, J=8.1 Hz, 1H), 7.46 (d, J=5.2 Hz, 2H), 7.32 (d, J=8.1 Hz, 1H), 7.19 (s, 1H), 2.44 (s, 3H), 2.35 (s, 3H). ¹³C NMR (101 MHz, CDCl₃): δ 140.4, 139.7, 137.8, 136.9, 135.0, 134.8, 133.2, 130.1, 129.2, 127.0, 125.5, 123.8, 21.5, 20.9.

### Embodiment 17: Synthesis of 6-methoxy-1-m-methoxyphenylbenzo[d][1,3,2]thiaselenazole-1-ketone (Compound 17)

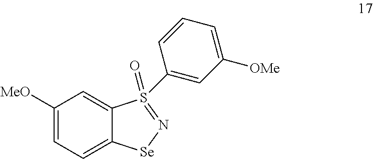 [SMILES: COc1cccc(S2(=O)=N[Se]c3ccc(OC)cc32)c1]

A reference was made to Embodiment 1 for the preparation method, with the difference that 22.1 mg of the target compound was obtained using iminodi-m-methoxyphenyl-λ⁶-sulfone as a substrate, with the yield of 62%. ¹H NMR (400 MHz, CDCl₃): δ 7.55 (d, J=7.8 Hz, 1H), 7.50-7.44 (m, 3H), 7.18 (dd, J=8.1, 1.7 Hz, 1H), 7.13 (dd, J=8.7, 2.3 Hz, 1H), 6.85 (d, J=2.2 Hz, 1H), 3.86 (s, 3H), 3.75 (s, 3H). ¹³C NMR (101 MHz, CDCl₃): δ 160.2, 159.2, 141.8, 135.3, 131.8, 130.3, 124.7, 122.0, 121.1, 120.5, 114.1, 108.5, 56.0, 55.9.

### Embodiment 18: Synthesis of 6-chloro-1-m-chlorophenylbenzo[d][1,3,2]thiaselenazol-1-ketone (Compound 18)

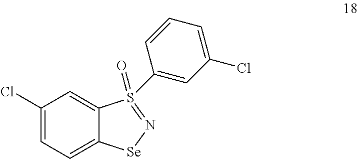 [SMILES: O=S1(c2cccc(Cl)c2)=N[Se]c2ccc(Cl)cc21]

A reference was made to Embodiment 1 for the preparation method, with the difference that 31.9 mg of the target compound was obtained using iminodi-m-chlorophenyl-λ⁶-sulfone as a substrate, with the yield of 88%. ¹H NMR (400 MHZ, CDCl₃): δ 7.94 (m, 1H), 7.89 (d, J=7.8 Hz, 1H), 7.68-7.64 (m, 1H), 7.60-7.52 (m, 2H), 7.47 (dd, J=8.5, 1.8 Hz, 1H), 7.36 (d, J=1.6 Hz, 1H). ¹³C NMR (101 MHz, CDCl₃): δ 141.8, 134.0, 135.8, 135.4, 134.5, 132.8, 132.3, 130.7, 129.8, 127.9, 125.4, 125.2.

### Embodiment 19: Synthesis of 7-methyl-1-o-methylphenylbenzo[d][1,3,2]thiaselenazole-1-ketone (Compound 19)

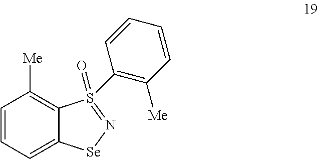 [SMILES: Cc1ccccc1S1(=O)=N[Se]c2cccc(C)c21]

A reference was made to Embodiment 1 for the preparation method, with the difference that 17.5 mg of the target compound was obtained using iminodi-o-methylphenyl-λ⁶-sulfone as a substrate, with the yield of 54%. ¹H NMR (400 MHZ, CDCl₃): δ 8.40 (d, J=7.9 Hz, 1H), 7.48 (m, 4H), 7.26 (d, J=7.8 Hz, 1H), 7.07 (d, J=7.0 Hz, 1H), 2.07 (s, 3H), 1.85 (s, 3H). ¹³C NMR (101 MHz, CDCl₃): δ 142.6, 140.5, 139.1, 137.0, 134.0, 133.0, 132.2, 131.1, 130.5, 128.4, 126.8, 121.8, 19.48, 17.89.

### Embodiment 20: Synthesis of 4,6-dimethyl-1-(3,5-dimethylphenyl)benzo[d][1,3,2]thiaselenazole-1-ketone (Compound 20)

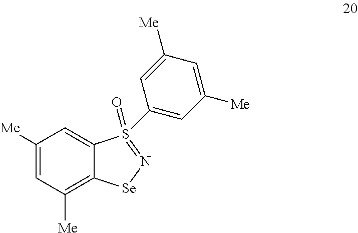 [SMILES: Cc1cc(C)cc(S2(=O)=N[Se]c3c(C)cc(C)cc32)c1]

A reference was made to Embodiment 1 for the preparation method, with the difference that 11.6 mg of the target compound was obtained using iminodi-3,5-dimethylphenyl-λ⁶-sulfone as a substrate, with the yield of 33%. ¹H NMR (400 MHZ, CDCl₃): δ 7.58 (s, 2H), 7.27 (s, 1H), 7.13 (s, 1H), 7.03 (s, 1H), 2.39 (s, 6H), 2.32 (s, 6H). ¹³C NMR (101 MHz, CDCl₃): 140.5, 139.4, 138.7, 138.0, 135.6, 134.6, 133.4, 133.3, 127.2, 122.8, 21.4, 21.3, 20.9.

### Embodiment 21: Synthesis of 1-(2-naphthyl)naphtho[2,3-d][1,3,2]thiaselenazole-1-ketone (Compound 21)

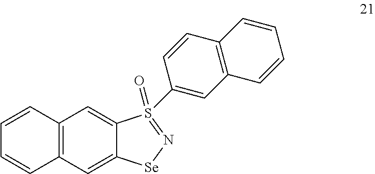 [SMILES: O=S1(c2ccc3ccccc3c2)=N[Se]c2cc3ccccc3cc21]

A reference was made to Embodiment 1 for the preparation method, with the difference that 12.0 mg of the target compound was obtained using iminodi-2-naphthyl-λ⁶-sulfone as a substrate, with the yield of 30%. ¹H NMR (400 MHZ, CDCl₃): δ 8.80 (s, 1H), 8.06 (d, J=6.0 Hz, 2H), 7.96 (m, 3H), 7.88 (d, J=8.8 Hz, 1H), 7.83 (d, J=8.4 Hz, 1H), 7.76 (d, J=8.3 Hz, 1H), 7.70 (t, J=7.4 Hz, 1H), 7.67-7.63 (m, 1H), 7.58 (t, J=7.5 Hz, 1H), 7.44 (t, J=7.5 Hz, 1H). ¹³C NMR (101 MHZ, CDCl₃): δ 136.8, 136.4, 135.6, 134.8, 134.7, 132.4, 131.8, 131.3, 129.83, 129.80, 129.7, 129.4, 129.3, 128.1, 127.9, 127.4, 126.6, 126.1, 124.5, 122.4.

### Embodiment Synthesis of 1-methylbenzo[d][1,3,2]thiaselenazole-1-ketone (Compound 22)

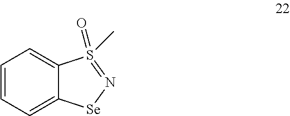

A reference was made to Embodiment 1 for the preparation method, with the difference that 24.3 mg of the target compound was obtained using imino(methyl) (phenyl)-λ⁶-sulfone as a substrate, with the yield of 62%. ¹H NMR (400 MHZ, CDCl₃): δ 7.78 (d, J=7.9 Hz, 1H), 7.64 (d, J=7.9 Hz, 1H), 7.56 (t, J=7.2 Hz, 1H), 7.45 (t, J=7.2 Hz, 1H), 3.56 (s, 3H). ¹³C NMR (101 MHZ, CDCl₃): δ 142.4, 133.5, 132.1, 126.5, 124.7, 124.6, 45.6.

### Embodiment 23: Synthesis of 5-methyl-1-methylbenzo[d][1,3,2]thiaselenazole-1-ketone (Compound 23)

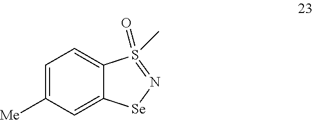

A reference was made to Embodiment 1 for the preparation method, with the difference that 23.7 mg of the target compound was obtained using imino(methyl) (p-methylphenyl)-λ⁶-sulfone as a substrate, with the yield of 48%. ¹H NMR (400 MHZ, CDCl₃): δ 7.65 (d, J=8.1 Hz, 1H), 7.41 (s, 1H), 7.24 (d, J=8.1 Hz, 1H), 3.52 (s, 3H), 2.46 (s, 3H). ¹³C NMR (101 MHz, CDCl₃): δ 143.4, 142.8, 131.1, 127.9, 124.5, 124.4, 45.7, 21.7.

### Embodiment 24: Synthesis of 5-methoxy-1-methylbenzo[d][1,3,2]thiaselenazole-1-ketone (Compound 24)

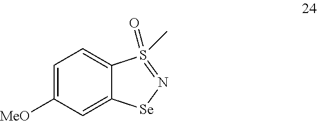

A reference was made to Embodiment 1 for the preparation method, with the difference that 22.0 mg of the target compound was obtained using imino(methyl) (p-methoxyphenyl)-λ⁶-sulfone as a substrate, with the yield of 42%. ¹H NMR (400 MHZ, CDCl₃): δ 7.64 (d, J=8.4 Hz, 1H), 7.03 (s, 1H), 6.95 (d, J=8.4 Hz, 1H), 3.88 (s, 3H), 3.49 (s, 3H). ¹³C NMR (101 MHz, CDCl₃): δ 163.0, 145.3, 126.0, 125.8, 115.0, 107.3, 56.0, 46.0.

### Embodiment 25: Synthesis of 5-fluoro-1-methylbenzo[d][1,3,2]thiaselenazole-1-ketone (Compound 25)

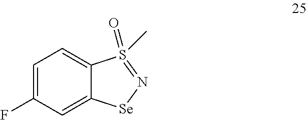

A reference was made to Embodiment 1 for the preparation method, with the difference that 23.6 mg of the target compound was obtained using imino(methyl) (p-fluorophenyl)-λ⁶-sulfone as a substrate, with the yield of 47%. ¹H NMR (400 MHZ, CDCl₃): δ 7.73 (dd, J=8.7, 4.6 Hz, 1H), 7.30 (dd, J=8.0, 2.0 Hz, 1H), 7.15 (td, J=8.4, 2.0 Hz, 1H), 3.55 (s, 3H). ¹³C NMR (101 MHz, CDCl₃): δ 165.0 (d, J=257.2 Hz), 145.9 (d, J=5.9 Hz), 129.9, 126.6 (d, J=10.5 Hz), 115.1 (d, J=25.0 Hz), 111.4 (d, J=25.7 Hz), 45.9. ¹⁹F NMR (376 MHz, CDCl₃): δ −106.3 (dd, J=12.2, 7.2 Hz).

### Embodiment 26: Synthesis of 5-chloro-1-methylbenzo[d][1,3,2]thiaselenazole-1-ketone (Compound 26)

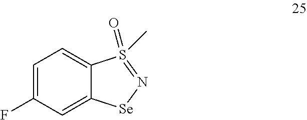

A reference was made to Embodiment 1 for the preparation method, with the difference that 27.2 mg of the target compound was obtained using imino(methyl) (p-chlorophenyl)-λ⁶-sulfone as a substrate, with the yield of 51%. ¹H NMR (400 MHZ, CDCl₃): δ 7.68 (d, J=8.4 Hz, 1H), 7.61 (s, 1H), 7.40 (d, J=8.3 Hz, 1H), 3.56 (s, 3H). ¹³C NMR (100 MHz, CDCl₃): δ 144.7, 139.3, 132.2, 127.1, 125.5, 124.4, 45.8.

### Embodiment 27: Synthesis of 5-bromo-1-methylbenzo[d][1,3,2]thiaselenazole-1-ketone (Compound 27)

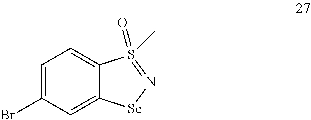

A reference was made to Embodiment 1 for the preparation method, with the difference that 26.7 mg of the target compound was obtained using imino(methyl) (p-bromophenyl)-λ⁶-sulfone as a substrate, with the yield of 43%. ¹H NMR (400 MHZ, CDCl₃): δ 7.79 (s, 1H), 7.61 (d, J=8.3 Hz, 1H), 7.55 (d, J=8.4 Hz, 1H), 3.55 (s, 3H). ¹³C NMR (101 MHz, CDCl₃): δ 144.9, 132.6, 129.9, 127.7, 127.3, 125.6, 45.7.

### Embodiment 28: Synthesis of 5-methylformyl-1-methylbenzo[d][1,3,2]thiaselenazole-1-ketone (Compound 28)

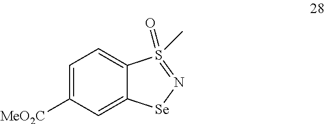

A reference was made to Embodiment 1 for the preparation method, with the difference that 35.6 mg of the target compound was obtained using imino(methyl) (p-methylformylphenyl)-λ⁶-sulfone as a substrate, with the yield of 61%. ¹H NMR (400 MHZ, CDCl₃): δ 8.32 (s, 1H), 8.07 (d, J=7.7 Hz, 1H), 7.83 (d, J=8.0 Hz, 1H), 3.98 (s, 3H), 3.62 (s, 3H). ¹³C NMR (101 MHz, CDCl₃): δ 165.4, 142.9, 136.4, 133.4, 127.6, 126.1, 124.4, 53.0, 45.4.

### Embodiment 29: Synthesis of 5-nitro-1-methylbenzo[d][1,3,2]thiaselenazole-1-ketone (Compound 29)

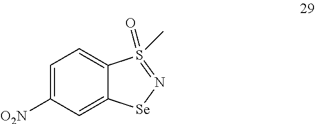

A reference was made to Embodiment 1 for the preparation method, with the difference that 19.5 mg of the target compound was obtained using iminod(methyl)(p-nitrophenyl)-λ⁶-sulfone as a substrate, with the yield of 35%, ¹H NMR (400 MHz, DMSO-d₆): δ 8.92 (s, 1H), 8.34 (d, J=8.6 Hz, 1H), 8.26 (d, J=8.5 Hz, 1H), 3.82 (s, 3H). ¹³C NMR (101 MHz, DMSO-d₆): δ 149.0, 144.2, 138.5, 125.7, 121.6, 44.4.

### Embodiment 30: Synthesis of 5-methyl-1-methylbenzo[d][1,3,2]thiaselenazole-1-ketone (Compound 30)

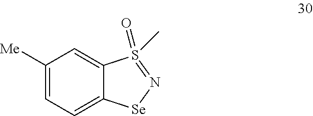

A reference was made to Embodiment 1 for the preparation method, with the difference that 31.7 mg of the target compound was obtained using imino(methyl) (m-methylphenyl)-λ⁶-sulfone as a substrate, with the yield of 64%. ¹H NMR (400 MHZ, CDCl₃): δ 7.58 (s, 1H), 7.50 (d, J=8.1 Hz, 1H), 7.39 (d, J=8.1 Hz, 1H), 3.54 (s, 3H), 2.48 (s, 3H). ¹³C NMR (101 MHz, CDCl₃): δ 138.8, 137.1, 133.7, 133.7, 124.7, 124.3, 45.5, 21.0.

### Embodiment 31: Synthesis of 6-methoxy-1-methylbenzo[d][1,3,2]thiaselenazole-1-ketone (Compound 31)

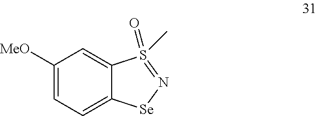

A reference was made to Embodiment 1 for the preparation method, with the difference that 28.9 mg of the target compound was obtained using imino(methyl) (m-methoxyphenyl)-λ⁶-sulfone as a substrate, with the yield of 55%, ¹H NMR (400 MHZ, DMSO-d₆): δ 7.82 (d, J=8.7 Hz, 1H), 7.71 (s, 1H), 7.23 (d, J=8.7 Hz, 1H), 3.85 (s, 3H), 3.69 (s, 3H). ¹³C NMR (101 MHz, DMSO-d₆): δ 158.7, 135.5, 131.6, 126.2, 120.5, 108.0, 56.0, 44.5.

### Embodiment 32: Synthesis of 6-chloro-1-methylbenzo[d][1,3,2]thiaselenazole-1-ketone (Compound 32)

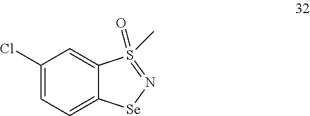

A reference was made to Embodiment 1 for the preparation method, with the difference that 27.7 mg of the target compound was obtained using imino(methyl) (m-chlorophenyl)-λ⁶-sulfone as a substrate, with the yield of 52%. ¹H NMR (400 MHZ, DMSO-d₆): δ 7.64 (d, J=8.4 Hz, 1H), 7.03 (s, 1H), 6.95 (d, J=8.4 Hz, 1H), 3.88 (s, 3H), 3.49 (s, 3H). ¹³C NMR (101 MHz, DMSO-d₆): δ 140.6, 136.3, 131.3, 131.0, 127.3, 124.3, 44.4.

### Embodiment 33: Synthesis of 7-chloro-1-methylbenzo[d][1,3,2]thiaselenazole-1-ketone (Compound 33)

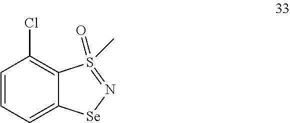

A reference was made to Embodiment 1 for the preparation method, with the difference that 26.6 mg of the target compound was obtained using imino(methyl) (o-chlorophenyl)-λ⁶-sulfone as a substrate, with the yield of 66%. ¹H NMR (400 MHZ, CDCl₃): δ 7.58 (dd, J=7.5, 3.8 Hz, 2H), 7.37 (t, J=7.8 Hz, 1H), 3.75 (s, 3H). ¹³C NMR (101 MHz, CDCl₃): δ 145.3, 134.0, 133.0, 130.7, 123.7, 119.5, 48.2.

### Embodiment 34: Synthesis of Compound 34

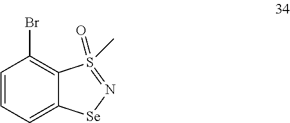

A reference was made to Embodiment 1 for the preparation method, with the difference that 43.5 mg of the target compound was obtained using imino(methyl) (o-bromophenyl)-λ⁶-sulfone as a substrate, with the yield of 70%. ¹H NMR (400 MHZ, CDCl₃): δ 7.54 (d, J=7.8 Hz, 1H), 7.47 (t, J=7.7 Hz, 1H), 7.38 (d, J=7.6 Hz, 1H), 3.75 (s, 3H). ¹³C NMR (101 MHz, CDCl₃): δ 145.3, 133.1, 131.7, 131.5, 127.3, 123.1, 47.9.

### Embodiment 35: Synthesis of 1-methylnaphtho[2,3-d][1,3,2]thiaselenazole-1-ketone (Compound 35)

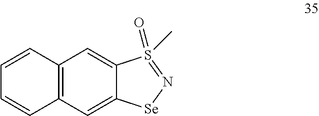

A reference was made to Embodiment 1 for the preparation method, with the difference that 18.1 mg of the target compound was obtained using imino(methyl) (naphthyl)-λ⁶-sulfone as a substrate, with the yield of 32%. ¹H NMR (400 MHZ, CDCl₃): δ 8.32 (s, 1H), 8.02-7.95 (m, 2H), 7.85 (d, J=8.3 Hz, 1H), 7.65 (t, J=7.5 Hz, 1H), 7.55 (t, J=7.5 Hz, 1H), 3.61 (s, 3H). ¹³C NMR (101 MHz, CDCl₃): δ 135.1, 134.9, 131.2, 129.6, 129.5, 127.5, 126.9, 125.0, 123.0, 44.8.

### Embodiment 36: Synthesis of 1-ethylbenzo[d][1,3,2]thiaselenazole-1-ketone (Compound 36)

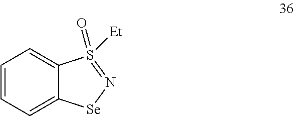 [SMILES: CCS1(=O)=N[Se]c2ccccc21]

A reference was made to Embodiment 1 for the preparation method, with the difference that 36.7 mg of the target compound was obtained using imino (ethyl) (phenyl)-λ⁶-sulfone as a substrate, with the yield of 74%. ¹H NMR (400 MHZ, CDCl₃): δ 7.72 (d, J=7.9 Hz, 1H), 7.64 (d, J=8.0 Hz, 1H), 7.56 (t, J=7.5 Hz, 1H), 7.43 (t, J=7.4 Hz, 1H), 3.80-3.67 (m, 2H), 1.28 (dd, J=9.4, 5.3 Hz, 3H). ¹³C NMR (101 MHz, CDCl₃): δ 143.7, 132.1, 131.1, 126.4, 125.2, 124.6, 52.5, 9.0.

### Embodiment 37: Synthesis of 1-cyclopropylbenzo[d][1,3,2]thiaselenazole-1-ketone (Compound 37)

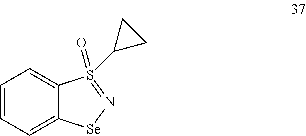 [SMILES: O=S1(C2CC2)=N[Se]c2ccccc21]

A reference was made to Embodiment 1 for the preparation method, with the difference that 27.4 mg of the target compound was obtained using imino (cyclopropyl) (phenyl)-λ⁶-sulfone as a substrate, with the yield of 81%. ¹H NMR (400 MHZ, CDCl₃): δ 7.77 (d, J=7.9 Hz, 1H), 7.61 (d, J=8.0 Hz, 1H), 7.54 (t, J=7.5 Hz, 1H), 7.42 (t, J=7.5 Hz, 1H), 2.82 (tt, J=7.7, 4.6 Hz, 1H), 1.38-1.16 (m, 4H). ¹³C NMR (101 MHz, CDCl₃): δ 142.7, 133.8, 131.9, 126.3, 125.2, 124.5, 33.2, 6.8, 5.0.

### Embodiment 38: Synthesis of 1-chloromethylbenzo[d][1,3,2]thiaselenazole-1-ketone (Compound 38)

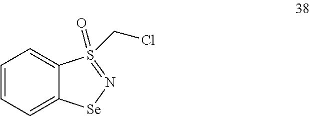 [SMILES: O=S1(CCl)=N[Se]c2ccccc21]

A reference was made to Embodiment 1 for the preparation method, with the difference that 41.9 mg of the target compound was obtained using imino (chloromethyl) (phenyl)-λ⁶-sulfone as a substrate, with the yield of 78%. ¹H NMR (400 MHZ, CDCl₃): δ 7.85 (d, J=8.0 Hz, 1H), 7.68-7.62 (m, 2H), 7.52-7.46 (m, 1H), 5.12 (d, J=12.6 Hz, 1H), 4.86 (d, J=12.6 Hz, 1H). ¹³C NMR (100 MHz, CDCl₃): δ 145.0, 133.1, 128.1, 126.8, 126.7, 124.6, 60.5.

### Embodiment 39: Synthesis of (R)-1-chloromethylbenzo[d][1,3,2]thiaselenazole-1-ketone (Compound (R)-1)

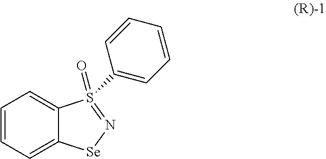 [SMILES: O=[S@@]1(c2ccccc2)=N[Se]c2ccccc21]

[Cp*Rh(MeCN)₃(SbF₆)₂] (5 mol %), AgF (0.25 mmol, 2.5 equiv.), selenium (0.3 mmol, 3 equiv.), sulfoximine (0.1 mmol, 1 equiv.), and CPA3 (30 mol %) were added into a 10 mL sealed tube, dissolved in trichloroethylene (1 ml), and then let react for 36 h in an oil bath at 60° C. After the reaction was completed, ethyl acetate was added into the reaction tube for dilution; a reaction solution was transferred to a round-bottomed flask and concentrated to obtain a crude product, which was then separated by column chromatography and eluted with an eluent of PE:EA=3:1 to obtain 25.1 mg of a product, with the yield of 85%, e.r.=6:94, [α]_{D}²⁵=−174.0 (c=0.1, CH₂Cl₂).

### Embodiment 40: Synthesis of(S)-1-chloromethylbenzo[d][1,3,2]thiaselenazole-1-ketone (Compound (R)-1)

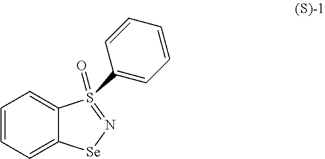 [SMILES: O=[S@]1(c2ccccc2)=N[Se]c2ccccc21]

A reference was made to Embodiment 39 for the preparation method, with the difference that 41.9 mg of the target compound was obtained by taking(S)-CPA3 as a catalyst, with the yield of 84%, e.r.=95:5, and [α]²⁵=+176.0 (c=0.1, CH₂Cl₂).

### Embodiment 41: Synthesis of (R)-5-methyl-1-(p-methylphenyl)benzo[d][1,3,2]thiaselenazole-1-ketone (Compound (R)-2)

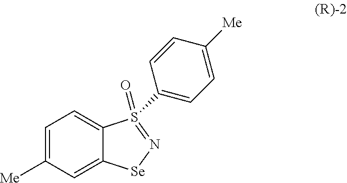 [SMILES: Cc1ccc([S@]2(=O)=N[Se]c3cc(C)ccc32)cc1]

A reference was made to Embodiment 39 for the preparation method, with the difference that 22.8 mg of the target compound was obtained by taking iminodi-p-methylphenyl-λ⁶-sulfone as a substrate, with the yield of 71%, e.r.=8:92, and [α]²⁵=−146.0 (c=0.1, CH₂Cl₂).

### Embodiment 42: Synthesis of (R)-5-propyl-1-(p-propylphenyl)benzo[d][1,3,2]thiaselenazole-1-ketone (Compound (R)-3)

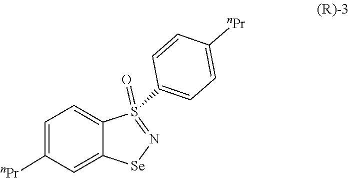

A reference was made to Embodiment 39 for the preparation method, with the difference that 31.3 mg of the target compound was obtained by taking iminodi-p-propylphenyl-λ⁶-sulfone as a substrate, with the yield of 83%, e.r.=13:87, and [α]²⁵=−98.0 (c=0.1, CH₂Cl₂).

### Embodiment 43: Synthesis of (R)-5-tert-butyl-1-(p-tert-butylphenyl)benzo[d][1,3,2]thiaselenazole-1-ketone (Compound (R)-4)

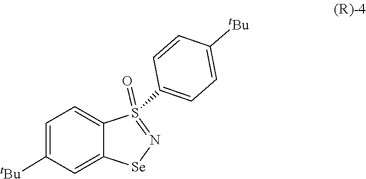 [SMILES: CC(C)(C)c1ccc([S@]2(=O)=N[Se]c3cc(C(C)(C)C)ccc32)cc1]

A reference was made to Embodiment 39 for the preparation method, with the difference that 19.2 mg of the target compound was obtained by taking iminodi-p-tert-butylphenyl-λ⁶-sulfone as a substrate, with the yield of 47%, e.r.=16:84, and [α]_{D}²⁵=−113.0 (c=0.1, CH₂Cl₂).

### Embodiment 44: Synthesis of (R)-5-methoxy-1-(p-methoxyphenyl)benzo[d][1,3,2]thiaselenazole-1-ketone (Compound (R)-5)

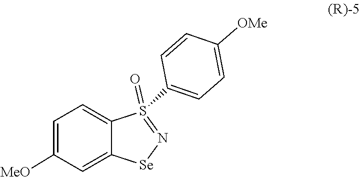 [SMILES: COc1ccc([S@]2(=O)=N[Se]c3cc(OC)ccc32)cc1]

A reference was made to Embodiment 39 for the preparation method, with the difference that 24.1 mg of the target compound was obtained by taking iminodi-p-methoxyphenyl-λ⁶-sulfone as a substrate, with the yield of 68%, e.r.=11:89, and [α]_{D}²⁵=−79.0 (c=0.1, CH₂Cl₂).

### Embodiment 45: Synthesis of (R)-5-fluoro-1-(4-fluorophenyl)benzo[d][1,3,2]thiaselenazole-1-ketone (Compound (R)-6)

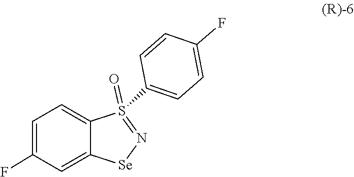 [SMILES: O=[S@@]1(c2ccc(F)cc2)=N[Se]c2cc(F)ccc21]

A reference was made to Embodiment 39 for the preparation method, with the difference that 16.6 mg of the target compound was obtained by taking iminodi-p-fluorophenyl-λ⁶-sulfone as a substrate, with the yield of 50%, e.r.=19:81, and [α]_{D}²⁵=−66.0 (c=0.1, CH₂Cl₂).

### Embodiment 46: Synthesis of (R)-5-chloro-1-(4-chlorophenyl)benzo[d][1,3,2]thiaselenazole-1-ketone (Compound (R)-7)

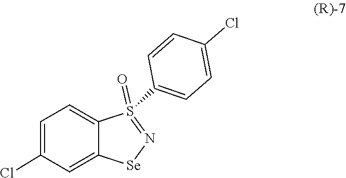 [SMILES: O=[S@@]1(c2ccc(Cl)cc2)=N[Se]c2cc(Cl)ccc21]

A reference was made to Embodiment 39 for the preparation method, with the difference that 21.3 mg of the target compound was obtained by taking iminodi-p-chlorophenyl-λ⁶-sulfone as a substrate, with the yield of 59%, e.r.=11:89, and [α]_{D}²⁵=−119.0 (c=0.1, CH₂Cl₂).

### Embodiment 47: Synthesis of (R)-5-bromo-1-(4-bromophenyl)benzo[d][1,3,2]thiaselenazole-1-ketone (Compound (R)-8)

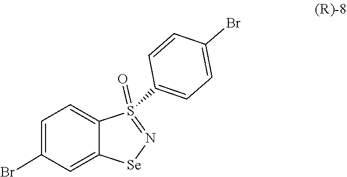 [SMILES: O=[S@@]1(c2ccc(Br)cc2)=N[Se]c2cc(Br)ccc21]

A reference was made to Embodiment 39 for the preparation method, with the difference that 37.0 mg of the target compound was obtained by taking iminodi-p-bromophenyl-λ⁶-sulfone as a substrate, with the yield of 82%, e.r.=13:87, and [α]_{D} ²⁵=−89.0 (c=0.1, CH₂Cl₂).

### Embodiment 48: Synthesis of (R)-5-trifluoromethyl-1-(4-trifluoromethylphenyl)benzo[d][1,3,2]thiaselenazole-1-ketone (Compound (R)-9)

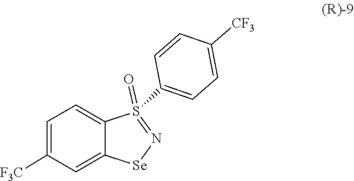 [SMILES: O=[S@@]1(c2ccc(C(F)(F)F)cc2)=N[Se]c2cc(C(F)(F)F)ccc21]

A reference was made to Embodiment 39 for the preparation method, with the difference that 17.2 mg of the target compound was obtained by taking iminodi-p-trifluoromethylphenyl-λ⁶-sulfone as a substrate, with the yield of 43%, e.r.=21:79, and [α]_{D} ²⁵=−117.0 (c=0.1, CH₂Cl₂).

### Synthesis of (R)-5-trifluoromethoxy-1-(4-Embodiment trifluoromethoxyphenyl)benzo[d][1,3,2]thiaselenazole-1-ketone (Compound (R)-10)

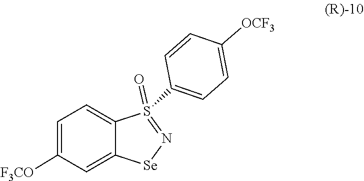 [SMILES: O=[S@@]1(c2ccc(OC(F)(F)F)cc2)=N[Se]c2cc(OC(F)(F)F)ccc21]

A reference was made to Embodiment 39 for the preparation method, with the difference that 10.9 mg of the target compound was obtained by taking iminodi-p-trifluoromethoxyphenyl-λ⁶-sulfone as a substrate, with the yield of 24%, e.r.=23:77, and [α]_{D}²⁵=−139.0 (c=0.1, CH₂Cl₂).

### Embodiment 50: Synthesis of (R)-5-nitro-1-(4-nitrophenyl)benzo[d][1,3,2]thiaselenazole-1-ketone (Compound (R)-11)

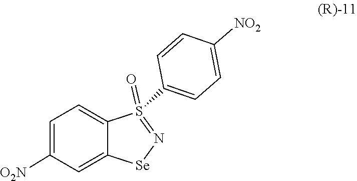 [SMILES: O=[N+]([O-])c1ccc([S@]2(=O)=N[Se]c3cc([N+](=O)[O-])ccc32)cc1]

A reference was made to Embodiment 39 for the preparation method, with the difference that 11.6 mg of the target compound was obtained by taking iminodi-p-nitrophenyl-λ⁶-sulfone as a substrate, with the yield of 30%, e.r.=15:85, and [α]_{D}²⁵=−287.0 (c=0.1, CH₂Cl₂).

### Embodiment 51: Synthesis of (R)-5-methylformyl-1-(4-methylformylphenyl)benzo[d][1,3,2]thiaselenazole-1-ketone (Compound (R)-12)

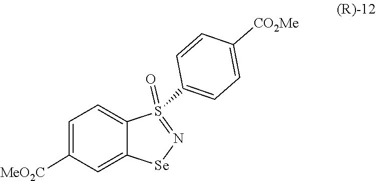 [SMILES: COC(=O)c1ccc([S@]2(=O)=N[Se]c3cc(C(=O)OC)ccc32)cc1]

A reference was made to Embodiment 39 for the preparation method, with the difference that 21.9 mg of the target compound was obtained by taking iminodi-p-methylformylphenyl-λ⁶-sulfone as a substrate, with the yield of 53%, e.r.=21:79, and [α]_{D}²⁵=−259.0 (c=0.1, CH₂Cl₂).

### Embodiment 52: Synthesis of (R)-5-trifluoromethanesulfonyl-1-(4-trifluoromethanesulfonylphenyl)benzo[d][1,3,2]thiaselenazole-1-ketone (Compound (R)-13)

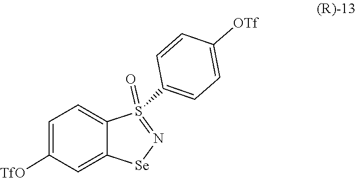 [SMILES: O=S(=O)(Oc1ccc([S@]2(=O)=N[Se]c3cc(OS(=O)(=O)C(F)(F)F)ccc32)cc1)C(F)(F)F]

A reference was made to Embodiment 39 for the preparation method, with the difference that 18.5 mg of the target compound was obtained by taking iminodi-p-trifluoromethanesulfonylphenyl-λ⁶-sulfone as a substrate, with the yield of 31%, e.r.=12:88, and [α]_{D}²⁵=−78.0 (c=0.1, CH₂Cl₂).

### Embodiment 53: Synthesis of (R)-5-p-toluenesulfonyloxy-1-(4-p-toluenesulfonyloxyphenyl)benzo[d][1,3,2]thiaselenazole-1-ketone (Compound (R)-14)

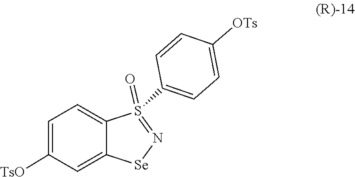 [SMILES: Cc1ccc(S(=O)(=O)Oc2ccc([S@]3(=O)=N[Se]c4cc(OS(=O)(=O)c5ccc(C)cc5)ccc43)cc2)cc1]

A reference was made to Embodiment 39 for the preparation method, with the difference that 16.7 mg of the target compound was obtained by taking iminodi-p-toluenesulfonyloxyphenyl-λ⁶-sulfone as a substrate, with the yield of 26%, e.r.=12:88, and [α]_{D} ²⁵=−115.0 (c=0.1, CH₂Cl₂).

### Embodiment 54: Synthesis of (R)-5-phenyl-1-biphenylbenzo[d][1,3,2]thiaselenazolw-1-ketone (Compound (R)-15)

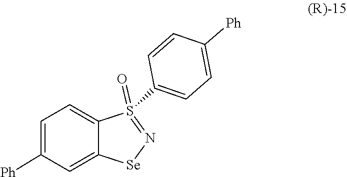 [SMILES: O=[S@@]1(c2ccc(-c3ccccc3)cc2)=N[Se]c2cc(-c3ccccc3)ccc21]

A reference was made to Embodiment 39 for the preparation method, with the difference that 19.5 mg of the target compound was obtained by taking iminodi-biphenyl-λ⁶-sulfone as a substrate, with the yield of 44%, e.r.=21:79, and [α]_{D}²⁵=−130.0 (c=0.1, CH₂Cl₂).

### Embodiment 55: Synthesis of (R)-6-methyl-1-m-methylphenylbenzo[d][1,3,2]thiaselenazole-1-ketone (Compound (R)-16)

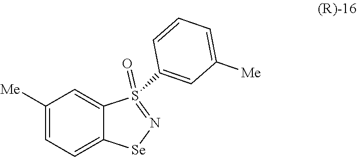 [SMILES: Cc1cccc([S@]2(=O)=N[Se]c3ccc(C)cc32)c1]

A reference was made to Embodiment 39 for the preparation method, with the difference that 16.1 mg of the target compound was obtained by taking iminodi-biphenyl-λ⁶-sulfone as a substrate, with the yield of 50%, e.r.=83:17, and [α]_{D}²⁵=−222.0 (c=0.1, CH₂Cl₂).

### Embodiment 56: Synthesis of (R)-6-methoxy-1-m-methoxyphenylbenzo[d][1,3,2]thiaselenazole-1-ketone (Compound (R)-17)

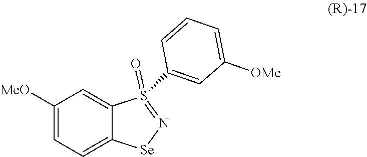 [SMILES: COc1cccc([S@]2(=O)=N[Se]c3ccc(OC)cc32)c1]

A reference was made to Embodiment 39 for the preparation method, with the difference that 7.8 mg of the target compound was obtained by taking iminodi-m-methoxyphenyl-λ⁶-sulfone as a substrate, with the yield of 22%, e.r.=21:79, and [α]_{D} ²⁵=−136.0 (c=0.1, CH₂Cl₂).

### Embodiment 57: Synthesis of (R)-6-chloro-1-m-chlorophenylbenzo[d][1,3,2]thiaselenazole-1-ketone (Compound (R)-18)

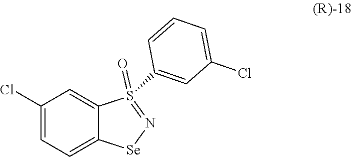 [SMILES: O=[S@@]1(c2cccc(Cl)c2)=N[Se]c2ccc(Cl)cc21]

A reference was made to Embodiment 39 for the preparation method, with the difference that 15.3 mg of the target compound was obtained by taking iminodi-m-chlorophenyl-λ⁶-sulfone as a substrate, with the yield of 43%, e.r.=15:85, and [α]_{D}²⁵=−191.0 (c=0.1, CH₂Cl₂).

### Embodiment 58: Synthesis of (R)-7-methyl-1-O-methylphenylbenzo[d][1,3,2]thiaselenazole-1-ketone (Compound (R)-19)

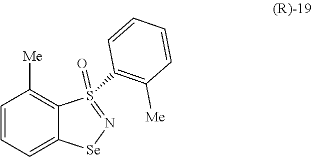 [SMILES: Cc1ccccc1[S@]1(=O)=N[Se]c2cccc(C)c21]

A reference was made to Embodiment 39 for the preparation method, with the difference that 7.7 mg of the target compound was obtained by taking iminodi-o-methylphenyl-λ⁶-sulfone as a substrate, with the yield of 24%, e.r.=23:77, and [α]_{D}²⁵=−144.0 (c=0.1, CH₂Cl₂).

### Embodiment 59: Synthesis of (R)-4,6-dimethyl-1-(3,5-dimethylphenyl)benzo[d][1,3,2]thiaselenazole-1-ketone (Compound (R)-21)

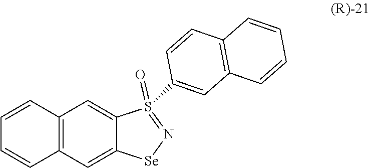 [SMILES: O=[S@@]1(c2ccc3ccccc3c2)=N[Se]c2cc3ccccc3cc21]

A reference was made to Embodiment 39 for the preparation method, with the difference that 12.0 mg of the target compound was obtained by taking iminodi-2-naphthyl-λ⁶-sulfone as a substrate, with the yield of 31%, e.r.=18:82, and [α]_{D} ²⁵=−158.0 (c=0.1, CH₂Cl₂).

### Embodiment 60: Synthesis of 1,2-bis(2-(phenylsulfonylimino)phenyl)diselenide (Compound 39)

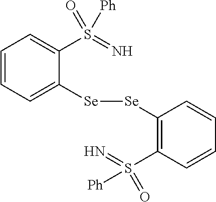 [SMILES: N=S(=O)(c1ccccc1)c1ccccc1[Se][Se]c1ccccc1S(=N)(=O)c1ccccc1]

The methanol (0.7 mL) solution of the compound 1 (0.2 mmol, 1.0 equiv.) prepared in Embodiment 1 was added into a sealed tube in an N₂ atmosphere. Hydrazine monohydrate (10 μL, 0.2 mmol, dissolved in 0.2 mL of methanol solution) was added via a syringe, which was then rinsed with 0.1 mL of methanol. The reaction mixture was stirred for 1 h in an oil bath at 65° C. Then, the mixture was poured into water and extracted three times with dichloromethane. Organic layers were combined, washed with saline solution (10 mL) and dried with anhydrous sodium sulfate. Then, the reaction mixture was concentrated and purified by column chromatography (eluent: PE/EA=3/1) to obtain the compound 39 at the yield of 91% (53.9 mg). ¹H NMR (400 MHz, CDCl₃): δ 8.15-8.12 (m, 6H), 7.62-7.56 (m, 2H), 7.52-7.50 (m, 4H), 7.44 (d, J=7.9 Hz, 2H), 7.29 (t, J=7.4 Hz, 2H), 7.09-7.01 (m, 2H), 3.51 (brs, 2H). ¹³C NMR (101 MHz, CDCl₃): δ 141.6, 141.5, 140.8, 140.7, 133.5, 133.0, 131.8, 131.7, 131.1, 131.0, 130.5, 129.0, 128.3, 127.2.

### Embodiment 61: Synthesis of methyl(2-(phenylsulfonylanilino)phenyl)selenide (Compound 40)

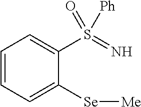 [SMILES: C[Se]c1ccccc1S(=N)(=O)c1ccccc1]

The compound 1 (0.2 mmol, 1.0 equiv.) and tetrahydrofuran solvent were added into a 10 mL Schlenk tube in an N₂ atmosphere. Then, magnesium methyl bromide (1.0 M tetrahydrofuran solution, 0.6 mL, 0.6 mmol) was then added dropwise at 0° C. The mixture was stirred for 2 h at room temperature, then poured into water and extracted three times with dichloromethane. Organic layers were combined, washed with saline solution (10 mL) and dried with anhydrous sodium sulfate. After evaporation of volatile matters, the resulting mixture was purified by column chromatography (eluent: PE/EA=3/1) to obtain the compound 40, with a yield of 92% (57.1 mg). ¹H NMR (400 MHz, CDCl₃): δ 8.23 (d, J=7.8 Hz, 1H), 8.11 (d, J=8.1 Hz, 2H), 7.56-7.50 (m, 1H), 7.46 (t, J=7.4 Hz, 2H), 7.43-7.37 (m, 1H), 7.34-7.29 (m, 2H), 3.05 (brs, 1H), 2.18 (s, 3H). ¹³C NMR (101 MHz, CDCl₃): δ 141.0, 140.8, 135.1, 132.9, 132.8, 130.9, 128.8, 128.7, 128.4, 125.4, 7.6.

### Embodiment 62: Synthesis of Se-(2-(N-benzoylphenylsulfonylimino)phenyl)benzoselenate (Compound 41)

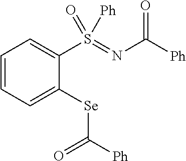 [SMILES: O=C(N=S(=O)(c1ccccc1)c1ccccc1[Se]C(=O)c1ccccc1)c1ccccc1]

Sodium borohydride (0.24 mmol, 1.2 equiv.) and ethanol (1 mL) were added into a 10 mL dry Schlenk tube in an N₂ atmosphere. Then, the compound 1 (0.2 mmol, 1.0 equiv.) was added to the above solution at 0° C. and stirred for 1 hour, benzoyl chloride (0.3 mmol, 1.5 equiv.) was then added, and the resulting mixture was heated to room temperature to allow for reaction for 12 h. At the end of the reaction, the mixture was poured into water (10 mL) and was extracted three times with methylene chloride (10 mL) to obtain a product. Organic layers were combined, washed with brine (10 mL), dried with anhydrous sodium sulfate, and concentrated under reduced pressure, and the resulting residue was separated and purified by column chromatography (eluent: DCM/EA=50/1) to obtain the compound 41, with the separation yield of 53% (53.3 mg). ¹H NMR (400 MHz, CDCl₃): δ 8.30-8.21 (m, 3H), 8.17-8.11 (m, 4H), 7.71-7.52 (m, 5H), 7.51-7.40 (m, 4H), 7.38-7.34 (m, 1H), 7.20 (t, J=6.8 Hz, 1H), 7.03 (t, J=7.6 Hz, 1H). ¹³C NMR (101 MHz, CDCl₃): δ 173.4, 171.9, 138.19, 138.17, 137.6, 137.5, 135.2, 134.2, 134.1, 133.9, 133.8, 132.7, 132.6, 131.9, 130.70, 130.66, 130.3, 129.93, 129.91, 129.53, 129.51, 129.40, 129.36, 129.35, 128.61, 128.57, 128.49, 128.3, 127.8, 127.7.

### Embodiment 63: Synthesis of 1-phenylbenzo[d][1,3,2]thiaselenazole1,3-dioxide (Compound 42)

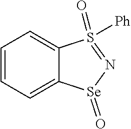 [SMILES: O=[Se]1N=S(=O)(c2ccccc2)c2ccccc21]

The chloroform solution (0.1 M) of the compound 1 (0.2 mmol, 1.0 equiv.) was added into a 10 mL Schlenk tube in an N₂ atmosphere. Then, 30% hydrogen peroxide (1.2 equiv.) was added dropwise at 0° C. The mixture was stirred for 12 h at room temperature, then poured into water and extracted three times with dichloromethane. Organic layers were combined, washed with saline solution (10 mL) and dried with anhydrous sodium sulfate. After evaporation of volatile matter, the resulting residue was purified by column chromatography (eluent: DCM/EA=10/1) to obtain the compound 42, with the separation yield of 90% (55.9 mg). This compound was a mixture of two inseparable diastereoisomers, and its dr value was determined to be 85:15 by ¹H-NMR analysis. ¹H NMR (400 MHZ, CDCl₃): δ 8.23 (d, J=7.5 Hz, 1.70H), 8.07 (d, J=7.6 Hz, 0.15H), 7.99 (t, J=10.4 Hz, 1.15H), 7.83 (t, J=7.2 Hz, 1H), 7.74-7.65 (m, 3H), 7.62 (t, J=7.3 Hz, 2H). ¹³C NMR (101 MHz, CDCl₃): δ 147.4, 140.6, 138.4, 135.1, 134.9 (minor isomer), 134.7 (minor isomer), 134.6, 133.0, 130.0, 129.4, 129.0 (minor isomer), 127.1 (minor isomer), 126.7, 126.2.

### Embodiment 64: Synthesis of 2-((2-(phenylsulfonylimino)phenyl)seleno)-1-(pyrimidin-2-yl)-1H-indole (Compound 43)

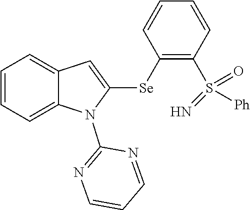 [SMILES: N=S(=O)(c1ccccc1)c1ccccc1[Se]c1cc2ccccc2n1-c1ncccn1]

The mixture of the compound 1 (0.1 mmol, 1.0 equiv.), 1-(pyrimidin-2-yl)-1H-indole (0.11 mmol, 1.1 equiv.), and [Cp*Rh(MeCN)₃(SbF₆)₂] (5 mol %) in 1,2-dichloroethane (0.5 mL) was stirred for 12 h at 100° C. Then, the mixture was diluted with ethyl acetate and filtered through a short silica gel column to remove metal residues. The reaction mixture was concentrated and then purified by column chromatography (eluent: PE/EA=3/1) to obtain the compound 43, with the separation yield of 64% (31.5 mg). ¹H NMR (400 MHZ, CDCl₃): δ 8.80 (d, J=8.4 Hz, 1H), 8.72 (d, J=4.6 Hz, 2H), 8.54 (s, 1H), 8.28-8.23 (m, 3H), 7.65-7.52 (m, 3H), 7.34 (t, J=7.7 Hz, 1H), 7.23 (t, J=7.4 Hz, 1H), 7.12-7.04 (m, 4H), 6.93 (d, J=7.8 Hz, 1H), 3.40 (brs, 1H). ¹³C NMR (101 MHz, CDCl₃): δ 158.4, 157.3, 141.5, 140.4, 136.2, 135.5, 133.3, 132.91, 132.86, 132.5, 131.0, 130.6, 128.9, 128.5, 126.0, 124.7, 122.9, 120.4, 117.0, 116.5, 104.8.

### Embodiment 65: Synthesis of 3-((2-(phenylsulfonylanilino)phenyl)seleno)-1H-indole (Compound 44)

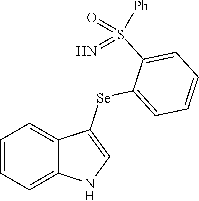 [SMILES: N=S(=O)(c1ccccc1)c1ccccc1[Se]c1c[nH]c2ccccc12]

The compound 1 (0.1 mmol, 1.0 equiv.), 1H-indole (0.12 mmol, 1.2 equiv.), and B(C₆F₅)₃ (5 mol %) in 1,2-dichloroethane solution (1.0 mL) were stirred for 2 h at room temperature; and at the end of the reaction, the solvent was removed and the residue was purified by column chromatography (eluent: PE/EA-3/1) to obtain the compound 44, with the separation yield of 89% (36.5 mg). ¹H NMR (400 MHZ, CDCl₃): δ 9.37 (brs, 1H), 8.23 (d, J=7.5 Hz, 2H), 8.17 (d, J=7.7 Hz, 1H), 7.58-7.53 (m, 1H), 7.49 (t, J=6.8 Hz, 2H), 7.40 (d, J=8.1 Hz, 1H), 7.34 (s, 1H), 7.19-7.11 (m, 2H), 7.01-6.91 (m, 4H), 3.13 (brs, 1H). ¹³C NMR (101 MHZ, CDCl₃): δ 141.2, 139.7, 136.82, 136.76, 133.0, 132.8, 132.6, 130.7, 130.5, 129.6, 128.9, 128.4, 125.7, 122.8, 120.7, 119.8, 111.8, 98.2.

### Embodiment 66: Synthesis of 2-(1-((((2-(phenylsulfonyl)phenyl) selanyl)thio)methyl)cyclopropyl) acetic acid (Compound 45)

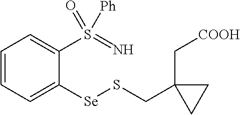 [SMILES: N=S(=O)(c1ccccc1)c1ccccc1[Se]SCC1(CC(=O)O)CC1]

In a nitrogen atmosphere, the dichloromethane solution of the compound 1 (0.1 mmol, 1.0 equiv.) and montelukast intermediate (0.12 mmol, 1.2 equiv., dissolved in 1 mL of dichloromethane) was added to a 10 mL Schlenk tube. Then, the reaction mixture was stirred for 12 hours at room temperature; at the end of the reaction, the solvent was removed under reduced pressure; and the resulting mixture was purified by column chromatography (eluent: DCM/EA/AcOH=10/1/0.1) to obtain the compound 45, with the separation yield of 95% (41.9 mg). ¹H NMR (400 MHZ, CDCl₃): δ 8.21 (d, J=7.9 Hz, 1H), 8.10-8.07 (m, 3H), 7.57-7.45 (m, 4H), 7.36 (t, J=7.4 Hz, 1H), 5.53 (brs, 2H), 2.87 (q, J=13.5 Hz, 2H), 2.53-2.42 (m, 2H), 0.45-0.41 (m, 4H). ¹³C NMR (101 MHZ, CDCl₃): δ 177.9, 141.8, 140.1, 134.1, 133.3, 133.2, 130.8, 129.4, 129.1, 128.0, 127.0, 46.5, 39.7, 18.2, 12.6, 12.5.

### Embodiment 67: Synthesis of (2-(((2-(phenylsulfonyl)phenyl)seleno)sulfo)propionyl)glycine (Compound 46)

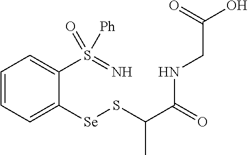 [SMILES: CC(S[Se]c1ccccc1S(=N)(=O)c1ccccc1)C(=O)NCC(=O)O]

A reference was made to Embodiment 66 for the preparation method, with the difference that the compound 46 was obtained by taking tiopronin as a substrate, with the separation yield of 90%. This compound was a mixture of two inseparable diastereoisomers, and its dr value was determined to be 1:1 by 1H-NMR analysis. ¹H NMR (400 MHZ, CDCl₃): δ 8.16-8.02 (m, 3H), 7.99 (d, J=7.6 Hz, 1H), 7.57-7.43 (m, 4H), 7.34 (t, J=7.2 Hz, 1H), 6.95-6.91 (m, 1H), 6.08 (brs, 3H), 3.81-3.73 (m, 1H), 3.65-3.59 (m, 1H), 3.54-3.49 (m, 1H), 1.40 (d, J=6.8 Hz, 1.5H), 1.37 (d, J=6.8 Hz, 1.5H). ¹³C NMR (101 MHz, CDCl₃): δ 172.5, 172.4, 172.3, 141.2, 139.5, 133.6, 133.24, 133.18, 133.1, 131.9, 131.8, 130.8, 130.6, 129.51, 129.45, 129.35, 129.1, 128.3, 127.93, 127.88, 127.5, 127.3, 47.8, 47.6, 41.6, 18.31, 18.26.

### Embodiment 68: Synthesis of ((2S)-2-methyl-3-((2-(phenylsulfonyl)phenyl)seleno)sulfo)propionyl)-L-proline (Compound 47)

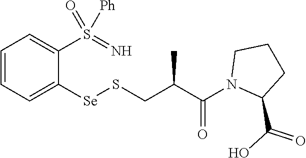 [SMILES: C[C@H](CS[Se]c1ccccc1S(=N)(=O)c1ccccc1)C(=O)N1CCC[C@H]1C(=O)O]

A reference was made to Embodiment 66 for the preparation method, with the difference that the compound 47 was obtained by taking captopril as a substrate, with the separation yield of 60%. This compound was a mixture of two inseparable diastereoisomers, and its dr value was determined to be 1:1 by 1H-NMR analysis. ¹H NMR (400 MHz, DMSO-d₆): δ 8.15-7.93 (m, 4H), 7.67-7.54 (m, 4H), 7.50 (t, J=7.4 Hz, 1H), 5.85 (s, 0.5H), 5.77 (s, 0.5H), 4.16 (d, J=8.4 Hz, 1H), 4.09 (d, J=8.4 Hz, 1H), 3.18-3.13 (m, 0.5H), 2.98-2.91 (m, 1H), 2.80-2.64 (m, 1H), 2.60-2.50 (m, 1H), 2.24-2.18 (m, 0.5H), 2.00-1.58 (m, 4H), 1.43-1.37 (m, 0.5H), 1.01-0.94 (m, 0.5H), 0.91 (d, J=6.6 Hz, 1.5H), 0.84 (d, J=6.6 Hz, 1.5H). ¹³C NMR (101 MHZ, DMSO-d₆): δ 173.3, 171.7, 171.4, 142.3, 140.4, 133.5, 133.3, 133.2, 132.9, 132.8, 130.5, 129.9, 129.6, 129.0, 128.9, 127.7, 127.5, 127.32, 127.28, 58.2, 58.1, 45.9, 45.3, 37.5, 37.0, 28.5, 28.3, 24.1, 23.8, 16.7, 16.6.

### Embodiment 69: Synthesis of N-(tert-butoxycarbonyl)-S-((2-(phenylsulfonyl)phenyl)seleno)-L-cysteine (Compound 48)

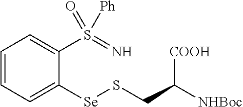 [SMILES: CC(C)(C)OC(=O)N[C@@H](CS[Se]c1ccccc1S(=N)(=O)c1ccccc1)C(=O)O]

A reference was made to Embodiment 66 for the preparation method, with the difference that the compound 48 was obtained by taking Boc-L-Cys as a substrate, with the separation yield of 92%. This compound was a mixture of two inseparable diastereoisomers, and its dr value was determined to be 1:1 by 1H-NMR analysis. ¹H NMR (400 MHZ, CDCl₃): δ 8.15 (d, J=7.6 Hz, 1H), 8.04 (d, J=6.9 Hz, 2H), 8.00 (d, J=7.6 Hz, 1H), 7.49-7.39 (m, 4H), 7.29 (t, J=7.8 Hz, 1H), 6.64 (brs, 2H), 5.78-5.56 (m, 1H), 4.35 (brs, 1H), 3.33-3.02 (m, 2H), 1.40 (s, 4.5H), 1.39 (s, 4.5H). ¹³C NMR (101 MHZ, CDCl₃): δ 175.1, 155.7, 141.9, 139.8, 133.5, 133.3, 130.8, 129.3, 129.2, 128.3, 127.8, 127.1, 80.3, 54.7, 54.5, 40.0, 39.7, 28.5.

### Embodiment 70: Synthesis of N-((tert-butoxycarbonyl)-L-alanine)-S-((2-(phenylsulfonyl)phenyl)seleno)-L-cysteine ethyl ester (Compound 49)

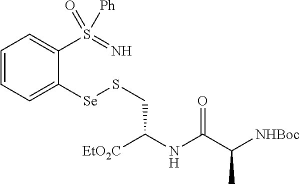 [SMILES: CCOC(=O)[C@H](CS[Se]c1ccccc1S(=N)(=O)c1ccccc1)NC(=O)[C@H](C)NC(=O)OC(C)(C)C]

A reference was made to Embodiment 66 for the preparation method, with the difference that the compound 49 was obtained by taking Boc-Ala-Cys-OEt as a substrate, with the separation yield of 80%. This compound was a mixture of two inseparable diastereoisomers, and its dr value was determined to be 1:1 by 1H-NMR analysis. ¹H NMR (400 MHZ, CDCl₃): δ 8.20-8.01 (m, 4H), 7.58-7.44 (m, 4H), 7.38 (t, J=7.4 Hz, 1H), 7.07 (brs, 1H), 5.30-5.10 (m, 1H), 4.89-4.73 (m, 1H), 4.34-4.15 (m, 3.5H), 3.33-3.05 (m, 2.5H), 1.47-1.44 (m, 9H), 1.32-1.23 (m, 6H). ¹³C NMR (101 MHz, CDCl₃): δ 172.6, 172.4, 170.1, 167.0, 155.6, 142.4, 142.3, 134.0, 133.5, 133.4, 133.3, 133.2, 133.0, 130.9, 130.8, 130.6, 129.23, 129.19, 129.0, 128.3, 128.0, 127.2, 80.3, 62.1, 53.2, 52.9, 50.1, 39.3, 39.0, 28.4, 18.2, 14.3, 14.2.

### Embodiment 71: Synthesis of 1-((N,4-dimethylphenyl)sulfonamide) vinyl N-(tert-butoxycarbonyl)-S-((2-(phenylsulfonylimino)phenyl)seleno)-L-cysteine (Compound 50)

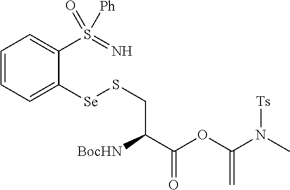 [SMILES: C=C(OC(=O)[C@H](CS[Se]c1ccccc1S(=N)(=O)c1ccccc1)NC(=O)OC(C)(C)C)N(C)S(=O)(=O)c1ccc(C)cc1]

A reference was made to Embodiment 66 for the preparation method, and the compound 50 was obtained with the separation yield of 85%. This compound was a mixture of two inseparable diastereoisomers, and its dr value was determined to be 1:1 by 1H-NMR analysis. 1H NMR (400 MHZ, CDCl₃): δ 8.20 (d, J=7.8 Hz, 1H), 8.14-7.96 (m, 3H), 7.78-7.67 (m, 2H), 7.58-7.45 (m, 4H), 7.40-7.30 (m, 3H), 5.45-5.16 (m, 1H), 4.93 (s, 1H), 4.63 (s, 1H), 4.51-4.40 (m, 1H), 3.29-3.03 (m, 3H), 2.98 (s, 1.5H), 2.96 (s, 1.5H), 2.43 (s, 3H), 1.46-1.44 (m, 9H). ¹³C NMR (101 MHz, CDCl₃): δ 170.5, 168.43, 168.39, 155.1, 155.0, 146.7, 145.1, 144.5, 142.2, 134.0, 136.1, 133.5, 133.3, 133.1, 130.8, 130.1, 129.7, 129.3, 129.2, 128.1, 127.8, 127.4, 127.3, 101.5, 80.5, 54.3, 54.2, 39.4, 39.1, 37.4, 33.2, 28.4, 25.1, 21.7.

### Embodiment 72: Synthesis of N-(tert-butoxycarbonyl)-S-((2-(phenylsulfonyl)phenyl)seleno)-L-cysteine-L-isoleucinate (Compound 51)

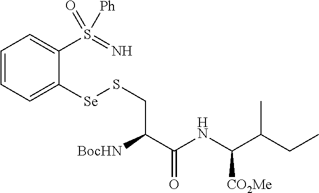 [SMILES: CCC(C)[C@H](NC(=O)[C@H](CS[Se]c1ccccc1S(=N)(=O)c1ccccc1)NC(=O)OC(C)(C)C)C(=O)OC]

A reference was made to Embodiment 66 for the preparation method, with the difference that the compound 51 was obtained by taking Boc-Cys-Ile-OMe as a substrate, with the separation yield of 86%. This compound was a mixture of two inseparable diastereoisomers, and its dr value was determined to be 1:1 by 1H-NMR analysis. ¹H NMR (400 MHZ, CDCl₃): δ 8.20 (d, J=7.7 Hz, 1H), 8.11-8.05 (m, 3H), 7.56-7.47 (m, 4H), 7.37 (t, J=7.4 Hz, 1H), 6.98 (brs, 1H), 5.37-5.25 (m, 1H), 4.59-4.52 (m, 1H), 4.32-4.16 (m, 1H), 3.73 (s, 3H), 3.21-3.02 (m, 2H), 1.95-1.86 (m, 1H), 1.46-1.37 (m, 11H), 1.24-1.11 (m, 1H), 0.92-0.88 (m, 6H). ¹³C NMR (101 MHz, CDCl₃): δ 172.0, 170.1, 155.6, 142.1, 140.1, 133.5, 133.2, 130.8, 129.3, 129.2, 127.8, 127.2, 80.7, 56.7, 54.8, 52.3, 39.0, 38.6, 37.9, 37.9, 28.3, 25.0, 15.5, 11.6.

### Embodiment 73: Synthesis of N₂-(tert-butoxycarbonyl)-N⁴-(2-(((2-(phenylsulfonyl)phenyl)seleno)sulfenyl)ethyl)-L-aspartyl-L-phenylalanine methyl ester (Compound 52)

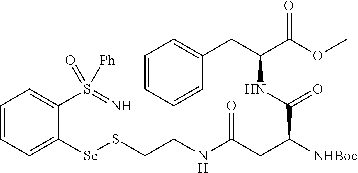 [SMILES: COC(=O)[C@H](Cc1ccccc1)NC(=O)[C@H](CC(=O)NCCS[Se]c1ccccc1S(=N)(=O)c1ccccc1)NC(=O)OC(C)(C)C]

A reference was made to Embodiment 66 for the preparation method, with the difference that the compound 52 was obtained by taking a aspartame derivative as a substrate, with the separation yield of 88%. This compound was a mixture of two inseparable diastereoisomers, and its dr value was determined to be 1:1 by 1H-NMR analysis. ¹H NMR (400 MHZ, CDCl₃): δ 8.18 (d, J=7.7 Hz, 1H), 8.09-8.06 (m, 3H), 7.56-7.39 (m, 5H), 7.35 (t, J=7.3 Hz, 1H), 7.29-7.20 (m, 3H), 7.14-7.11 (m, 2H), 6.70-6.62 (m, 1H), 6.18-6.12 (m, 1H), 4.75 (q, J=6.1 Hz, 1H), 4.48-4.40 (m, 1H), 3.64 (s, 3H), 3.39-3.21 (m, 2H), 3.07-3.05 (m, 2H), 2.82-2.70 (m, 3H), 2.53-2.49 (m, 1H), 1.40 (s, 9H). ¹³C NMR (101 MHZ, CDCl₃): δ 171.5, 171.1, 155.6, 141.9, 140.1, 135.8, 133.4, 133.3, 133.1, 130.7, 129.3, 129.0, 128.6, 127.8, 127.1, 80.2, 53.6, 52.3, 51.1, 39.4, 37.8, 37.3, 36.0, 28.3.

### Embodiment 74: Synthesis of 2-(acetoxymethyl)-6-(((2-(phenylsulfonyl)phenyl) selanyl)sulfenyl)tetrahydro-2H-pyran-3,4,5-triacetic acid (Compound 53)

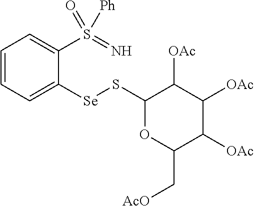 [SMILES: CC(=O)OCC1OC(S[Se]c2ccccc2S(=N)(=O)c2ccccc2)C(OC(C)=O)C(OC(C)=O)C1OC(C)=O]

A reference was made to Embodiment 66 for the preparation method, with the difference that the compound 53 was obtained by taking a D-glucopyranose derivative as a substrate, with the separation yield of 95%. This compound was a mixture of two inseparable diastereoisomers. ¹H NMR (400 MHZ, CDCl₃): δ 8.37 (d, J=8.0 Hz, 0.5H), 8.31 (d, J=8.0 Hz, 0.5H), 8.12 (t, J=7.6 Hz, 2H), 8.02 (d, J=7.8 Hz, 1H), 7.63-7.39 (m, 4H), 7.39-7.27 (m, 1H), 5.46-5.27 (m, 2H), 5.14-4.97 (m, 1H), 4.63-4.57 (m, 1H), 4.27-3.90 (m, 2H), 3.86-3.76 (m, 1H), 3.64-3.54 (m, 1H), 2.20-2.08 (m, 6H), 2.06-1.85 (m, 6H). ¹³C NMR (101 MHz, CDCl₃): δ 170.36, 170.30, 170.25, 170.1, 170.0, 169.6, 169.5, 169.4, 142.6, 142.4, 141.6, 140.7, 139.7, 139.6, 134.3, 134.1, 133.5, 133.3, 133.2, 133.0, 132.9, 131.7, 131.0, 130.7, 130.6, 130.5, 130.3, 129.2, 129.1, 128.9, 128.2, 127.8, 127.24, 127.17, 90.7, 88.9, 88.4, 74.8, 74.7, 71.8, 71.7, 68.6, 67.6, 67.2, 67.1, 61.5, 61.2, 60.9, 60.4, 21.1, 20.92, 20.85, 20.7, 20.64, 20.60, 20.57, 14.2.

### Embodiment 75: Synthesis of (2-((((3S,5S,8R,9S,10S,13R,14S,17R)-10,13-dimethyl-17-((R)-6-methyl heptane-2-yl) hexadecahydro-1H-cyclopentan[a]phenanthren-3-yl)sulfenyl)seleno)phenyl)(imino)(phenyl)-λ⁶-sulfonylketone (Compound 54)

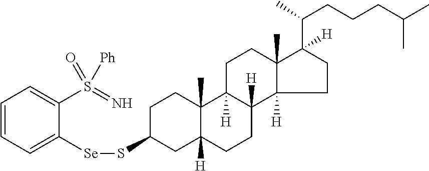

A reference was made to Embodiment 66 for the preparation method, with the difference that the compound 54 was obtained by taking a cholesterol derivative as a substrate, with the separation yield of 83%. This compound was a mixture of two inseparable diastereoisomers, and its dr value was determined to be 1:1 by 1H-NMR analysis. ¹H NMR (400 MHZ, CDCl₃): δ 8.13 (d, J=7.8 Hz, 1H), 8.04-8.02 (m, 3H), 7.49-7.34 (m, 4H), 7.27 (t, J=7.3 Hz, 1H), 3.03-2.99 (m, 1H), 2.80 (brs, 1H), 1.89 (d, J=11.6 Hz, 1H), 1.80-0.87 (m, 29H), 0.86-0.72 (m, 11H), 0.65-0.62 (m, 3H), 0.56 (s, 3H). ¹³C NMR (100 MHz, CDCl₃): δ 141.73, 141.65, 140.5, 134.5, 133.0, 130.62, 130.55, 130.1, 129.9, 128.92, 128.88, 128.11, 128.07, 126.8, 56.6, 56.3, 54.2, 48.6, 42.7, 40.3, 40.2, 40.1, 39.6, 36.40, 36.39, 36.3, 35.9, 35.51, 35.50, 33.4, 33.2, 33.1, 32.0, 28.39, 28.35, 28.3, 28.1, 27.0, 24.3, 24.0, 23.0, 22.7, 20.9, 18.8, 12.2, 11.8.

### Embodiment 76: Synthesis of Organic Selenium DNA-Encoded Compound Library

Hexafluoroisopropanol (HFIP, 13 μL),B(C₆F₅)₃ (1 μL, 40 mM in HFIP), and benzothiaselenazole-1-oxide (10 μL, 200 mM in HFIP) were added to DNA-labeled indole (4 μL, 500 mM, pH 4.2 PBS), and the resulting mixture was vortexed and allowed to stand for 6 h at room temperature. After incubation, 5 M NaCl solution (10 vol %) and ice ethanol (2.5-3 times volumes, ethanol stored at −20° C.) were added, vortexed, and incubated for at least 30 min at-80° C. The sample was centrifuged in a microcentrifuge at 12,000 rpm for 15 min at 4° C. to remove the supernatant. The resulting precipitate was re-dissolved in ddH₂O (5 μL) for LC-MS detection.

### Embodiment 77: Evaluation of Antiviral Activity In Vitro

### (1) SARS-CoV-2 Inhibition Experiment

Primate Vero E6 cells were seeded in a 96-well plate at a density of 1.5×10⁴ cells per well. After incubation overnight, the cells were pretreated for 2 h at 37° C. with the different concentrations of medicaments prepared in Embodiments 1-59. Before antiviral detection, 0.05 MOI of viruses were co-incubated with different concentrations of medicaments for 1 h, and 50 μL of the resulting mixture was transferred to medicament-pretreated Vero E6 cells and continued to incubate for 1 h. Then, the mixture was removed, added to a DMEM medium containing serially diluted medicaments and 2% FBS, and then incubated at 37° C. After 24 hours, 200 μL of 4% PFA was added to each well to fix the cells for 1 h, the fixation solution was discarded, and the cells were washed 3 times with PBS. Then, the cells were permeabilized with 0.2% Triton X-100 and blocked with 1% BSA for 30 min, respectively. SARS-CoV/SARS-CoV-2 Nucleocapsid Rabbit PAb antibodies (Sino Biological, cat #40143-T62) were added and incubated for 1 h at 37° C., followed by further incubation for 1 h at 37° C. in the dark by using Alexa Fluor 488 AffiniPure Donkey Anti-Rabbit IgG (h⁺L) fluorescent secondary antibodies (Alexa Fluor®488, Jackson, cat #711-545-152). The cells were washed 3 times with PBST, and protected from light at room temperature, during which nuclei were stained with DAPI for 15 min, and finally, the cells were scanned and analyzed with a Nexcelom Celigo imaging cytometer to calculate the virus inhibition rate.

### (2) M^{pro} Protein Inhibition Test

Based on the antiviral results in the Vero E6 cells, we further determined the inhibition levels of the benzothiaselenazole-1-oxide compound and its (R)-/(S)-isomer against the M^{pro} enzyme, with ebselen (EBS) as a positive control. The IC₅₀ value of the compounds against the M^{pro} proteins was determined by TR-FRET. The steps were as follows. 3CLpro (P132H mutation) was pre-incubated with the target compound for 30 min in a 96-well plate. An FRET-compatible peptide substrate MCA-AVLQSGFR-Lys (Dnp)-Lys-NH2 was added to initiate the reaction. Fluorescence measurements of 40 detection points were conducted every 10 seconds using a 340/405 excitation/emission filter on a multimode microplate reader (Thermo Scientific™ Varioskn™ LUX). The IC₅₀ values were calculated by fitting the curves with the four-parameter equation in GraphPad Prism.

The results indicated (FIG. 1):

1) excellent inhibitory activity was exhibited in the SARS-CoV-2 virus-infected primate Vero cells, with the effective inhibition rate of greater than 90% at the concentration of 10 μM, and moreover, the inhibitory activity of the compound 1 and its enantiomer against the M^{pro} protein of SARS-CoV-2 was superior to that of the ebselen (EBS) control group.

2) the benzothiaselenazole-1-oxide compound provided by the present invention exerted activity by covalently binding to the key Cys145 site in the M^{pro} protein of the SARS-CoV-2.

### Embodiment 78: Bio-Orthogonal Labeling of Trastuzumab and Imaging of HER² Receptors on the Cell Surface

### (1) Synthesis Method for Bifunctional Selenium Linker L1

1-(4-carboxyphenyl)-1λ4-benzo[d][1,3,2]thiophenazole-5-carboxylic acid 1-oxide (0.05 g, 0.13 mmol) and N-hydroxysulfosuccinimide sodium salt (NHSS) (0.062 g, 0.286 mmol) were placed in a round-bottomed flask containing 2.5 mL of DMF and 0.5 mL of water, and 1-(3-dimethylaminopropyl)-3-ethylcarbodiimide hydrochloride (EDCI) (0.055 g, 0.286 mmol) dissolved in 1 mL DMF solution was added dropwise at room temperature. The reaction mixture was stirred for 30 min at room temperature, and then 2-[2-(2-azidoethoxy) ethoxy]ethanolamine (0.045 g, 0.26 mmol) was slowly added to the reaction mixture and stirred for 24 h at 40° C. The resulting crude mixture was purified by HPLC to obtain a yellow solid L1 (0.043 g, with the yield of 48%).

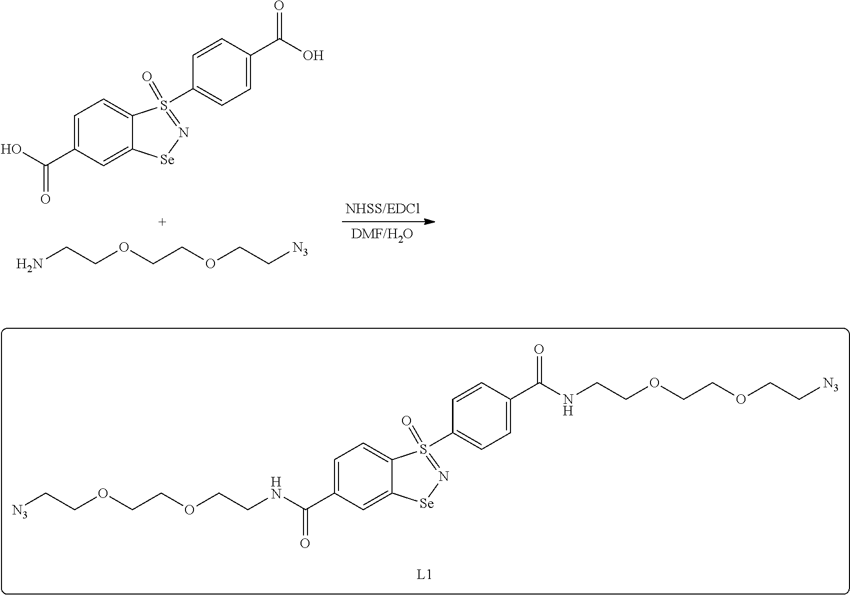 [SMILES: O=C(O)c1ccc(S2(=O)=N[Se]c3cc(C(=O)O)ccc32)cc1 ; [N-]=[N+]=NCCOCCOCCN ; [N-]=[N+]=NCCOCCOCCNC(=O)c1ccc(S2(=O)=N[Se]c3cc(C(=O)NCCOCCOCCN=[N+]=[N-])ccc32)cc1]

(2) Process of Labeling Trastuzumab with Bifunctional Selenium Linker L1

50 μL of 1 mg/mL trastuzumab (prepared with Hepes buffer (pH=8.0)) and 3.37 μL of 1 mM tris(2-carboxyethyl)phosphoric acid (TCEP) were added into a 1.5 mL tube, and let react for 2 h at room temperature to obtain reduced trastuzumab, and then, 1.69 μL of 10 mM L1 was added to let react for 12 h at room temperature. The conjugated C1 was purified using a Zeba™ spin desalting column (Thermo, 7K MWCO, 0.5 mL) to remove the excess reagent, and then characterized by UPLC-MS analysis. Afterwards, 3.37 μL of 1 mM Alexa Fluor®555 DIBO alkyne (Thermo Scientific) was added to C1 and let react for 4 h at room temperature, and the resultant was purified using the Zeba™ spin desalting column. The resulting conjugated product C2 was characterized by SDS-PAGE, in which 20 μL of C2 was transferred to another 1.5 mL tube, 4 μL of protein buffer (6×TRANS) was added, and the resulting mixture was heated for 5 min at 95° C. Another two control samples were prepared using the same method. For unreduced samples, 10 μL of trastuzumab (1 mg/mL) was mixed with 4 μL of 4×NuPAGE LDS sample buffer, and the samples were added to a protein precast gel and electrophoresed at 130 V for 1 hour. The buffer system is 1×MOPS SDS electrophoresis buffer (ABCONE). Fluorescence intensity was analyzed with a ChemiDoc MP imaging system. Then, 0.5% Coomassie brilliant blue was added, and the gel was washed 2-3 times and then analyzed.

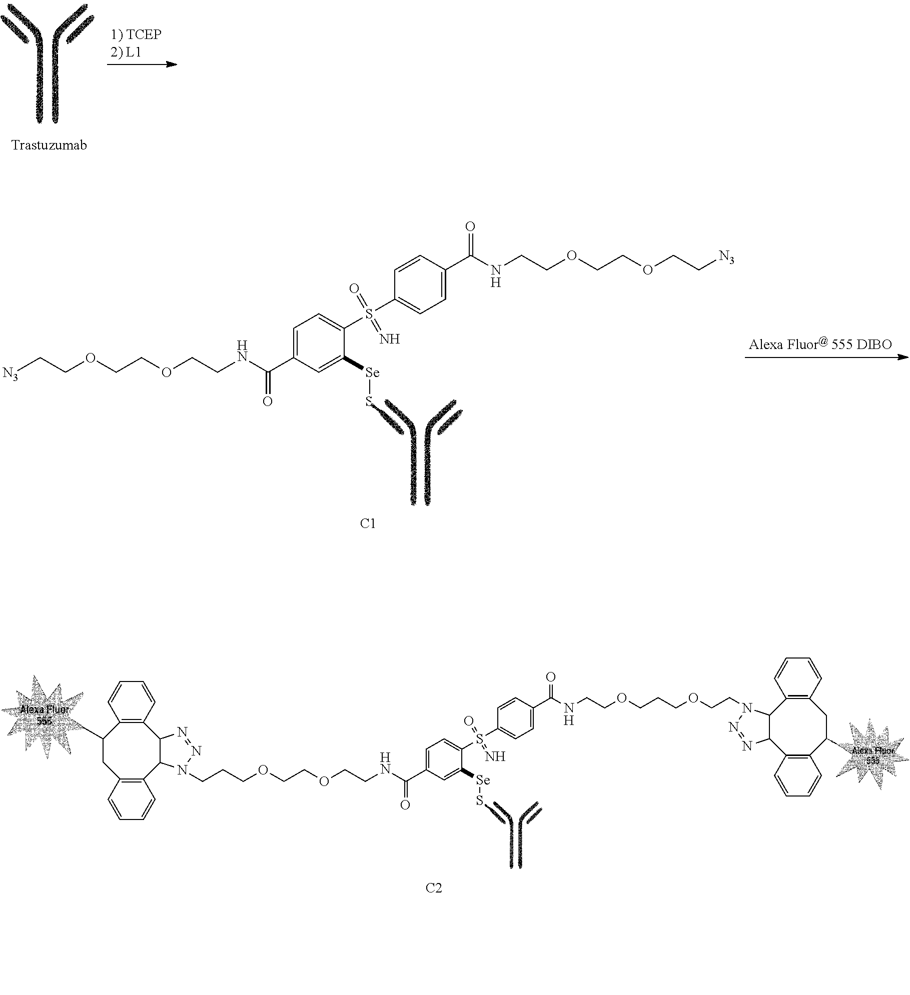

### (3) Procedures for Cell Staining and Imaging

a) 4×105 BT474 (HeR²⁺) or MCF-7 (HeR²−)) cells were seeded in a 24-well plate.
b) 1 ml of PBS was added and shaken gently for 5 min, and the cells were incubated with 150 μL of fixation buffer (BioLegend) for 20 min at room temperature in the dark.
c) The buffer was discarded, 1 ml of PBS was added to wash the plate.
d) The cells were incubated under the conditions shown in the table below, while being gently shaken at room temperature.

TABLE
Conditions for imaging HeR² receptors on the cell surface

| | NC1^{a} | | NC2^{a} | | Test | | | | | | | |
|---|---|---|---|---|---|---|---|---|---|---|---|---|
| Items | 2 h | | 2 h | | 0.5 h | | 1 h | | 1.5 h | | 2 h | |
| PBS (0.1% Triton) | 500 | μL | 500 | μL | 500 | μL | 500 | μL | 500 | μL | 500 | μL |

| | | | | | | | |
|---|---|---|---|---|---|---|---|
| C1 (1 mg/mL) | — | 1 | μL | — | — | — | — |

| | | | | | | | | | | |
|---|---|---|---|---|---|---|---|---|---|---|
| C2 (0.6 mg/mL)^{b} | — | — | 2 | μL | 2 | μL | 2 | μL | 2 | μL |

^{a}NC: negative control; ^{b}C2 was obtained by mixing equal amounts of L1 and Click-IT^{@} Alexa Fluor^{@} 555 DIBO alkyne (Thermo Scientific) for 12 h at room temperature. Afterwards, the mixture was conjugated to trastuzumab according to the procedures for labeling the trastuzumab with the bifunctional selenium linker L1.
e) 1 ml of PBS containing 0.1% Triton was added, shaken gently, and washed 2 times for 5 min each.
f) DAPI was diluted in PBS containing 0.1% Triton at a ratio of 1:2,000, 1 ml of DAPI solution was added per well, and the cells were incubated for 5 min at room temperature in the dark.

The results could be found in FIG. 2. Fluorescence microscopy images clearly showed that C2 could efficiently image the HER² receptors on the cell surface, and compared to negative MCF-7 (HER²⁻) cells, BT474 (HER²⁺) cells with high expression of HER² showed intense fluorescence.

1. A benzothiaselenazole-1-oxide compound of formula I: 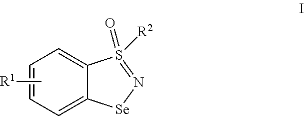
wherein R¹ is selected from any one of following groups: H, C1-C4 alkyl, halogen, phenyl, —CF₃, —OCF₃, —NO₂, —CO₂Me, —OTf, —OTs or —OMe; and R² is selected from any one of following groups: halogen, C1-C3 alkyl, cycloalkyl, chloroalkyl, phenyl or substituted phenyl or naphthyl.

2. The benzothiaselenazole-1-oxide compound according to claim 1, wherein the chloroalkyl is C1-C4 chloroalkyl.

3. The benzothiaselenazole-1-oxide compound according to claim 1, which is selected from following compounds:

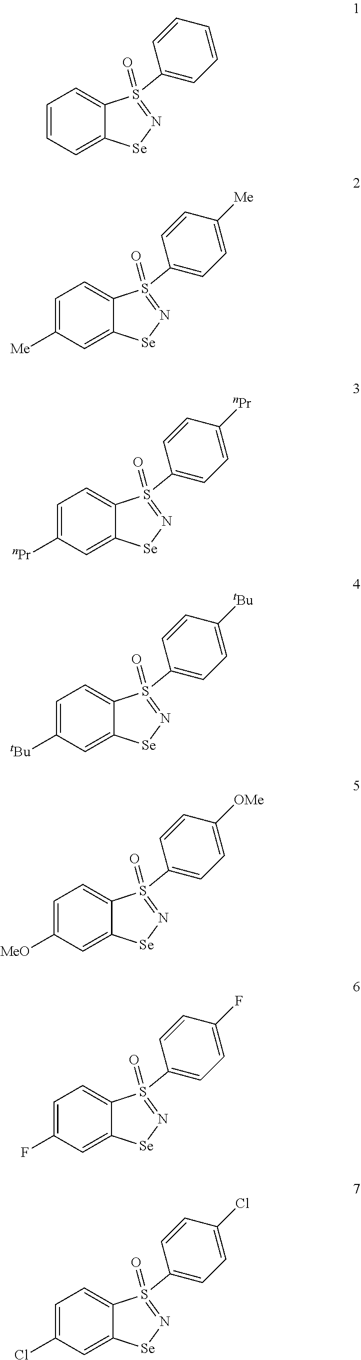

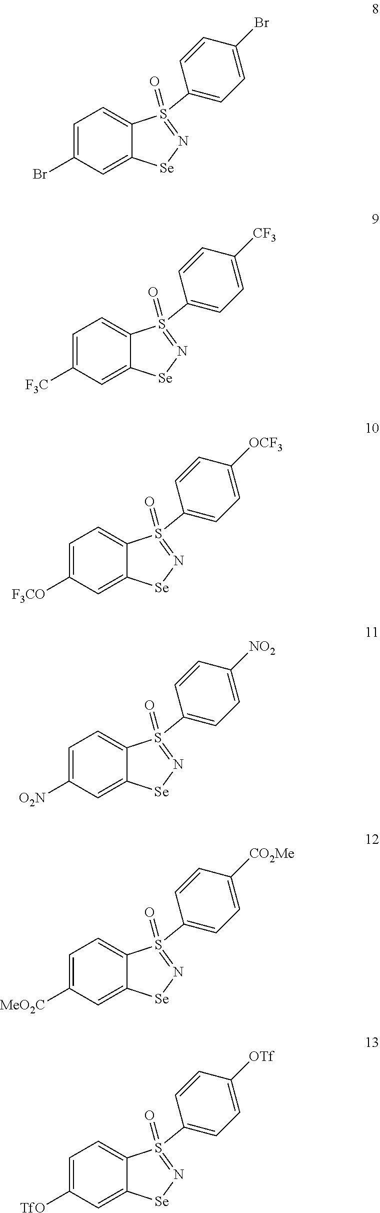 [SMILES: O=S1(c2ccc(Br)cc2)=N[Se]c2cc(Br)ccc21 ; O=S1(c2ccc(C(F)(F)F)cc2)=N[Se]c2cc(C(F)(F)F)ccc21 ; O=S1(c2ccc(OC(F)(F)F)cc2)=N[Se]c2cc(OC(F)(F)F)ccc21 ; O=[N+]([O-])c1ccc(S2(=O)=N[Se]c3cc([N+](=O)[O-])ccc32)cc1 ; COC(=O)c1ccc(S2(=O)=N[Se]c3cc(C(=O)OC)ccc32)cc1 ; O=S(=O)(Oc1ccc(S2(=O)=N[Se]c3cc(OS(=O)(=O)C(F)(F)F)ccc32)cc1)C(F)(F)F]

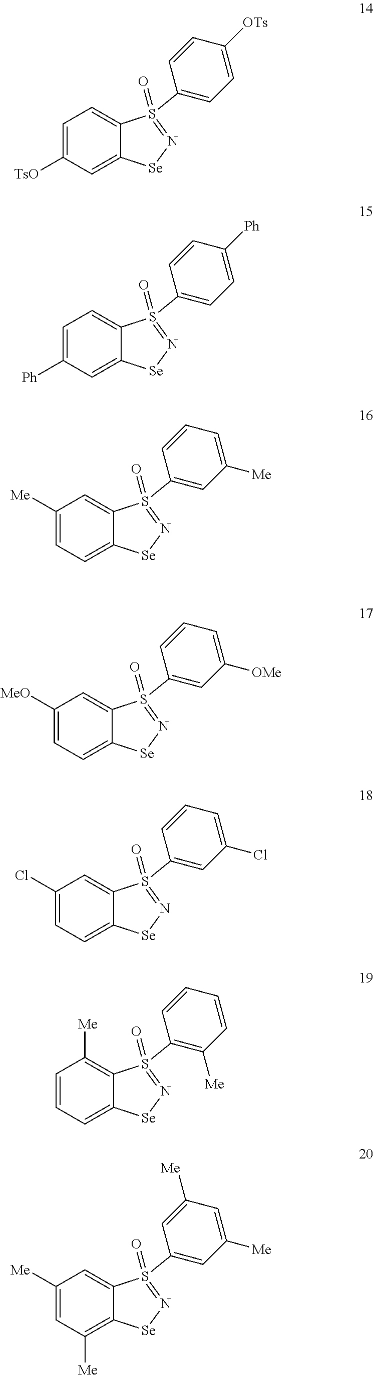

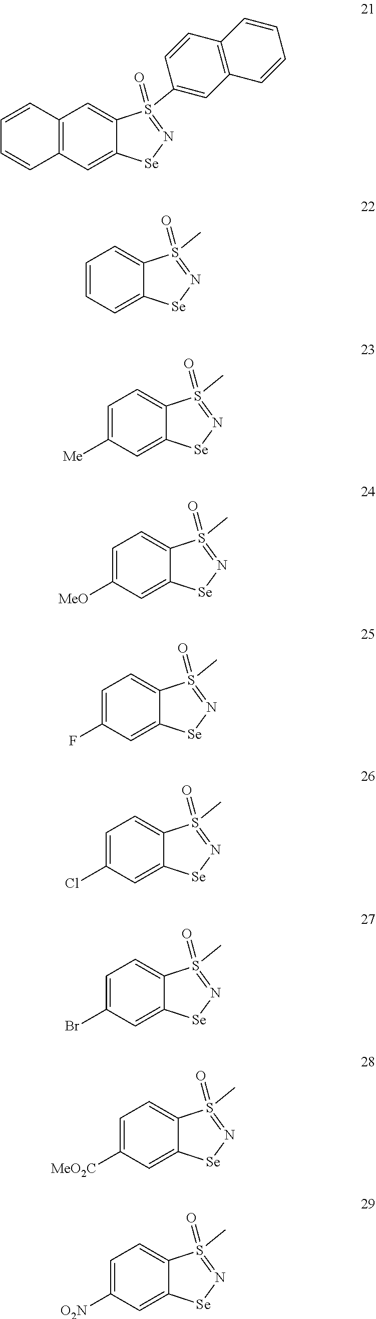

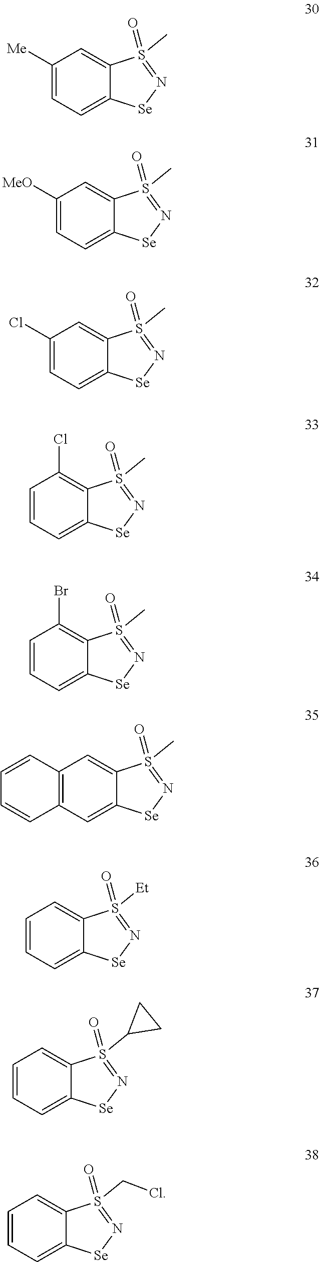

4. A synthesis method for a benzothiaselenazole-1-oxide compound of formula I, comprising steps of: reacting selenium with sulfoximine of formula III, which are taken as starting materials, under catalysis of rhodium at 90-110° C. to obtain the benzothiaselenazole-1-oxide compound: 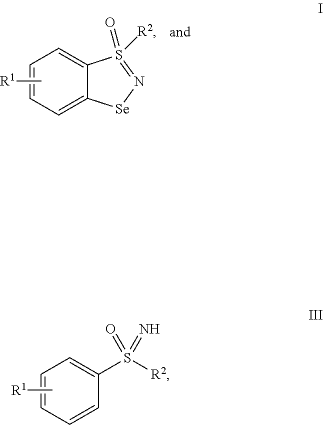
wherein, R¹ is selected from any one of following groups: H, C1-C4 alkyl, halogen, phenyl, —CF₃, —OCF₃, —NO₂, —CO₂Me, —OTf, —OTs or —OMe; and R² is selected from any one of following groups: halogen, C1-C3 alkyl, cycloalkyl, chloroalkyl, phenyl or substituted phenyl or naphthyl.

5. The synthesis method according to claim 4, in particular comprising following steps of: adding [Cp*Rh(MeCN)₃(SbF₆)₂], AgF, selenium, and sulfoximine into a sealed tube to allow for dissolution in dichloroethane, and letting react in an oil bath at 100° C., wherein a molar ratio of the AgF to the selenium to the sulfoximine is 2.5:(3-4):1.

6. An enantiomer of benzothiaselenazole-1-oxide compound of formula II: 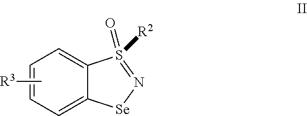
wherein R³ is selected from any one of following groups: H, C1-C4 alkyl, halogen, phenyl, —CF₃, —OCF₃, —NO₂, —CO₂Me, —OTf, —OTs or —OMe; and R⁴ is selected from any one of following groups: phenyl or substituted phenyl or naphthyl.

7. The enantiomer of benzothiaselenazole-1-oxide compound according to claim 6, which is selected from following compounds:

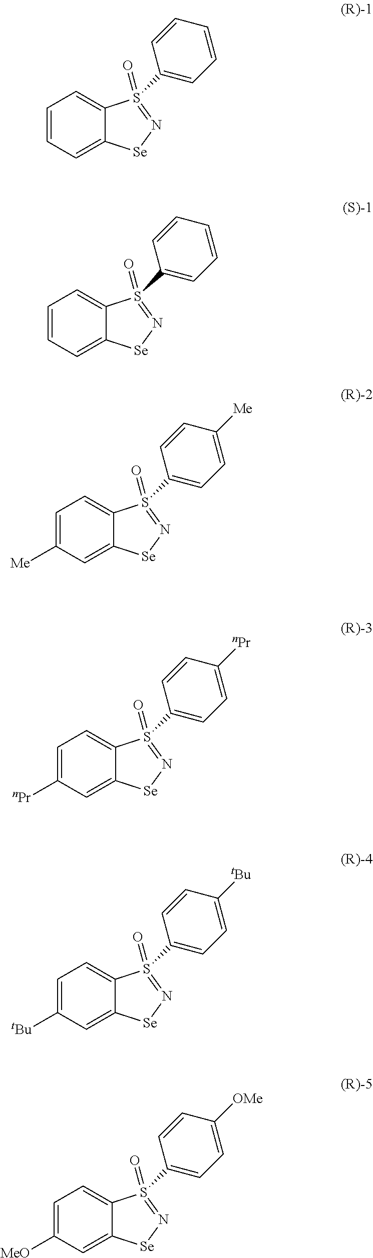

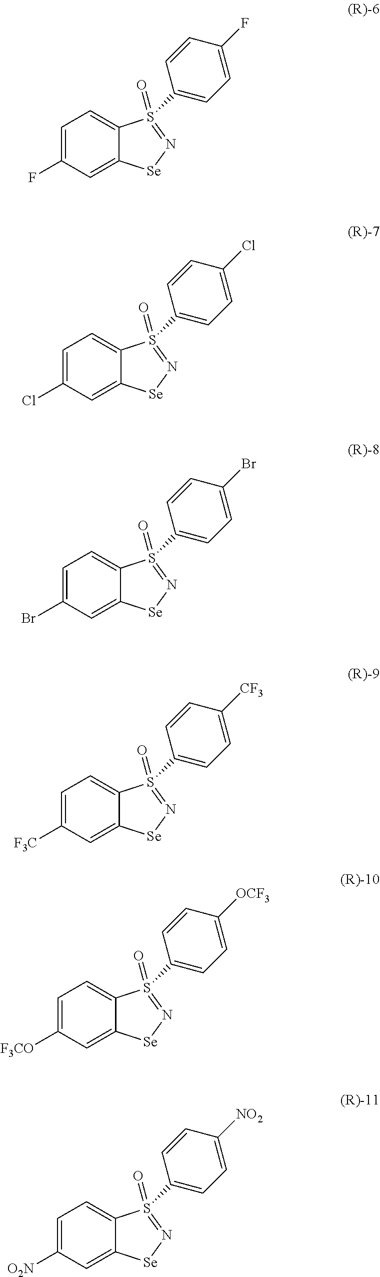 [SMILES: O=[S@@]1(c2ccc(F)cc2)=N[Se]c2cc(F)ccc21 ; O=[S@@]1(c2ccc(Cl)cc2)=N[Se]c2cc(Cl)ccc21 ; O=[S@@]1(c2ccc(Br)cc2)=N[Se]c2cc(Br)ccc21 ; O=[S@@]1(c2ccc(C(F)(F)F)cc2)=N[Se]c2cc(C(F)(F)F)ccc21 ; O=[S@@]1(c2ccc(OC(F)(F)F)cc2)=N[Se]c2cc(OC(F)(F)F)ccc21 ; O=[N+]([O-])c1ccc([S@]2(=O)=N[Se]c3cc([N+](=O)[O-])ccc32)cc1]

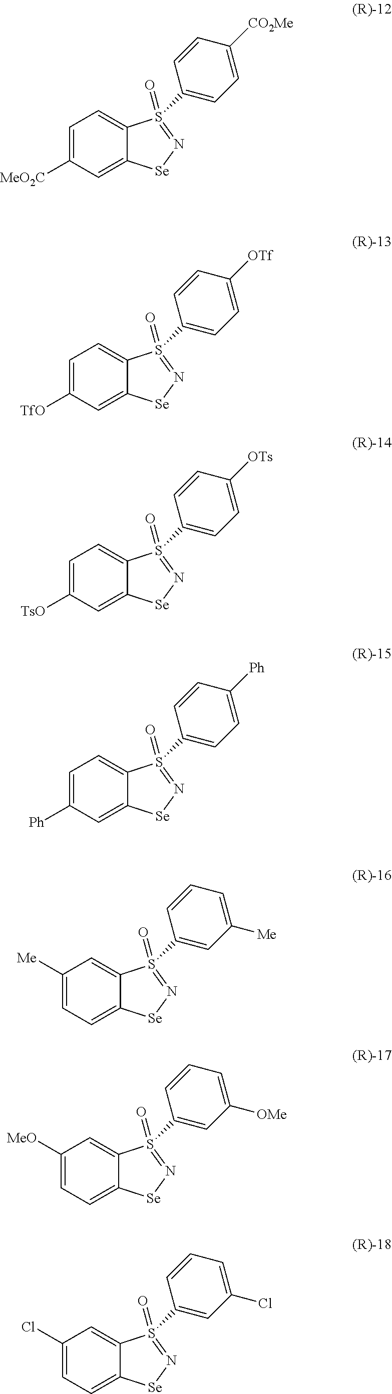

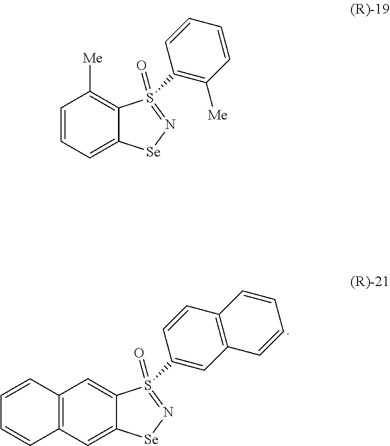 [SMILES: Cc1ccccc1[S@]1(=O)=N[Se]c2cccc(C)c21 ; O=[S@@]1(c2ccc3ccccc3c2)=N[Se]c2cc3ccccc3cc21]

8. A synthesis method for an enantiomer of benzothiaselenazole-1-oxide compound of formula II, comprising following steps of: subjecting selenium and sulfoximine of formula IV, which are taken as starting materials, to a direct C—H functionalization reaction by means of chiral phosphoric acid at 55-65° C. to obtain the enantiomer of benzothiaselenazole-1-oxide compound; 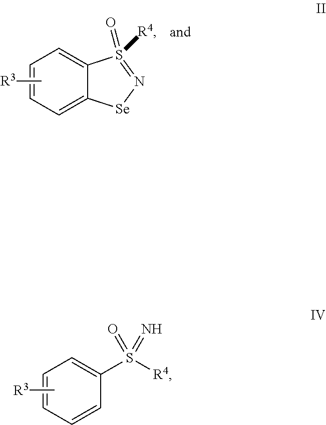
wherein R3 is selected from any one of following groups: H, C1-C4 alkyl, halogen, phenyl, —CF₃, —OCF₃, —NO₂, —CO₂Me, —OTf, —OTs or —OMe; and R⁴ is selected from any one of following groups: phenyl or substituted phenyl or naphthyl.

9. The synthesis method according claim 8, in particular comprising following steps of: adding [Cp*^{2ph}Rh(MeCN)₃(SbF₆)₂], AgF, selenium, sulfoximine, and chiral phosphoric acid into a sealed tube to allow for dissolution in trichloroethylene, and letting react in an oil bath at 60° C. to obtain the enantiomer of benzothiaselenazole-1-oxide compound, wherein a molar ratio of the AgF to the selenium to the sulfoximine is 2.5:(3-4):1.

10. Application of the benzothiaselenazole-1-oxide compound of formula I according to claim 1 in preparation of an anti-SARS-CoV-2 medicament.

11. Application of the benzothiaselenazole-1-oxide compound of formula I according to claim 1 in modification of sulfydryl-containing molecules based on Se—S covalent linkage, wherein the sulfydryl-containing molecules comprise amino acids, polypeptide and derivatives thereof, carbohydrates, and other pharmaceutical molecules.

12. Application of a trastuzumab bioconjugate of the benzothiaselenazole-1-oxide compound of formula I according to claim 1 in preparation of an imaging reagent for an HER² receptor on a cell surface.

13. Application of the benzothiaselenazole-1-oxide compound of formula I according to claim 1 their derivatives as a selenizing reagent, wherein a novel indole-selenium compound based on the benzothiaselenazole-1-oxide compound is obtained through reaction by taking indole or its derivative as a reactant and tris(pentafluorophenyl) borane as a catalyst.

14. A novel organic selenium compound based on the benzothiaselenazole-1-oxide compound obtained from the application according to claim 13, which has a structural formula as shown in formula V: 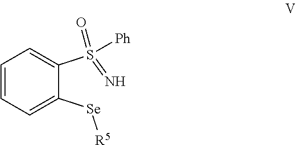
wherein R⁵ is selected from any one of following groups: alkyl, acyl, substituted indyl, substituted seleno, or a sulfydryl compound.

15. The novel organic selenium compound according to claim 14, wherein the substituted indyl comprises indole N, and an indole substrate with different substitutions at C2-C7 positions, and substituents comprise alkyl, alkoxy, ester, alkynyl, halogen, nitro, and an indole compound labeled with DNA tags at different sites.

16. The novel organic selenium compound according to claim 14, wherein the alkyl is a C1-C100 alkyl.

17. The novel organic selenium compound according to claim 16, wherein the alkyl is a C1-C10 alkyl.

18. Application of the enantiomer of benzothiaselenazole-1-oxide compound of formula II according to claim 4 in preparation of an anti-SARS-CoV-2 medicament.

19. Application of the enantiomer of benzothiaselenazole-1-oxide compound of formula II according to claim 4 in modification of sulfydryl-containing molecules based on Se—S covalent linkage, wherein the sulfydryl-containing molecules comprise amino acids, polypeptide and derivatives thereof, carbohydrates, and other pharmaceutical molecules.

20. Application of a trastuzumab bioconjugate of the enantiomer of benzothiaselenazole-1-oxide compound of formula II according to claim 4 in preparation of an imaging reagent for an HER² receptor on a cell surface.
